(19) United States
(12) Patent Application Publication
(10) Pub. No.: US 2025/0319054 A1
(43) Pub. Date: Oct. 16, 2025

(54) Synthetic Eicosanoid Analogues for the Treatment and Prevention of Diseases Associated with Increased GDF15 Plasma Concentration

(71) Applicant: OMEICOS Therapeutics GmbH, Berlin (DE)

(72) Inventors: Robert Fischer, Berlin (DE); Anne Konkel, Berlin (DE); Janine Lossie, Berlin (DE); Taro Inaba, Tokyo (JP); Wolf-Hagen Schunck, Berlin (DE)

(21) Appl. No.: 18/713,345

(22) PCT Filed: Nov. 25, 2022
(86) PCT No.: PCT/EP2022/083322

§ 371 Date: May 24, 2024

(30) Foreign Application Priority Data
Nov. 26, 2021 (EP) 21210906.0

(51) Int. Cl. A61K 31/221; A61K 31/165; A61K 31/167; A61K 31/195; A61K 31/196; A61K 31/351; A61K 31/365; A61K 31/40; A61K 31/41; A61K 31/4196; A61K 31/421; A61K 31/5375; A61P 3/06; A61P 3/10; A61P 9/04; A61P 9/06; A61P 9/10

(52) U.S. Cl. CPC A61K 31/221; A61K 31/165; A61K 31/167; A61K 31/195; A61K 31/196; A61K 31/351; A61K 31/365; A61K 31/40; A61K 31/41; A61K 31/4196; A61K 31/421; A61K 31/5375; A61P 3/06; A61P 3/10; A61P 9/04; A61P 9/06; A61P 9/10

(57) ABSTRACT

The present invention relates to compounds according to general formula (I) which are metabolically robust analogues of bioactive lipid mediators derived from omega-3 polyunsaturated fatty acids (n-3 PUFAs) for use in treating, reducing the risk of developing or preventing a disorder associated with an elevated GDF-15 plasma concentration.

The present invention relates to compounds according to general formula (I) which are metabolically robust analogues of bioactive lipid mediators derived from omega-3 polyunsaturated fatty acids (n-3 PUFAs) for use in treating or reducing the risk of developing or preventing a disorder associated with elevated GDF-15 plasma concentration.

### BACKGROUND OF THE INVENTION

Omega-6 and omega-3 polyunsaturated fatty acids (n-6 and n-3 PUFAs) are essential components of the mammalian diet. Biologically most important n-3 PUFAs are eicosapentaenoic acid (EPA, 20:5 n-3) and docosahexaenoic acid (DHA, 22:6 n-3). Dietary n-3 PUFAs have effects on diverse physiological processes impacting normal health and chronic disease, such as the regulation of plasma lipid levels, cardiovascular and immune function, inflammation, insulin action, and neuronal development and visual function.

Ingestion of n-3 PUFA will lead to their distribution to virtually every cell in the body with effects on membrane composition and function, eicosanoid synthesis, and signaling as well as the regulation of gene expression.

Simopoulos and colleagues summarized animal experiments and clinical intervention studies indicating that n-3-PUFAs have anti-inflammatory properties and, therefore, might be useful in the management of inflammatory and autoimmune diseases (Simopoulos A P. Omega-3 fatty acids in inflammation and autoimmune diseases. J Am. Coll. NutL 2L 495-505 (2002)).

One of the PUFAs most important biological roles is to supply precursors for the production of bioactive fatty acid metabolites that can modulate many functions. For instance, arachidonic acid (AA; 20:4, n-6) is metabolized by Cytochrome P450 (CYP) enzymes to several classes of oxygenated metabolites with potent biological activities. Major metabolites include 20-hydroxyeicosatetraenoic acid (20-HETE) and a series of regio- and stereoisomeric epoxyeicosatrienoic acids (EETs). CYP4A and CYP4F isoforms produce 20-HETE and CYP2C and CYP2J isoforms EETs.

It is known that EPA (20:5, n-3) and DHA (22:6, n-3) may serve as alternative substrates for AA-metabolizing CYP isoforms (Arnold C. et al., J Biol Chem. 2010 Oct. 22; 285(43):32720-33.; Fischer R. et al., J Lipid Res. 2014 Mar. 16; 55(6):1150-1164.). CYP2C and CYP2J subfamily members that epoxidize AA to EETs, metabolize EPA to epoxyeicosatetraenoic acids (EEQs), and DHA to epoxydocosapentaenoic acids (EDPs). The ω-3 double bond distinguishing EPA and DHA from AA is the preferred site of attack by most of the epoxygenases resulting in the formation of 17,18-EEQ and 19,20-EDP as main metabolites. CYP4A and CYP4F isoforms, hydroxylating AA to 20-HETE, metabolize EPA to 20-hydroxyeicosapentaenoic acid (20-HEPE) and DHA to 22-hydroxydocosahexaenoic acid (22-HDHA). CYP1A1, CYP2E1 and other isoforms converting AA predominantly to 19-HETE show pronounced ω-3 epoxygenase activities with EPA and DHA. Human CYP1A1 variants lead to differential eicosapentaenoic acid metabolite patterns. Cytochrome P450-dependent eicosapentaenoic acid metabolites are novel BK channel activators. A remarkable feature of CYP-dependent n-3 PUFA metabolism is the preferred epoxidation of the n-3 double bond, which distinguishes EPA and DHA from AA. The resulting metabolites—17,18-EEQ from EPA and 19,20-EDP from DHA—are unique in having no homolog within the series of AA products. In line with the substrate specificity of the CYP isoforms, dietary EPA/DHA supplementation causes a profound shift from AA- to EPA- and DHA-derived epoxy- and ω-hydroxy-metabolites in all major organs and tissues of the rat and presumably also in human.

EETs and 20-HETE play important roles in the regulation of various cardiovascular functions (Roman R J., Physiol Rev. 2002; 82:131-85). It has been shown that Ang II-induced hypertension is associated with a down-regulation of CYP-dependent AA metabolism (Kaergel et 1., Hypertension. 2002; 40:273-9) in a double-transgenic rat (dTGR) model of Ang II-induced hypertension and end-organ damage (Luft et al., Hypertension. 1999; 33:212-8). Recently, it has been shown that eicosapentaenoic acid (EPA) supplementation significantly reduced the mortality of dTGR (Theuer et al., Kidney Int. 2005; 67:248-58). Additionally, it has been shown that dTGR develop ventricular arrhythmias based on Ang II-induced electrical remodeling (Fischer et sl. Am J Physiol Heart Circ Physiol. 2007; 293:H1242-1253). Treatment of the dTGR rats with a PPAR-alpha activator strongly induced CYP2C23-dependent EET production and protected against hypertension and end-organ damage (Muller et al., Am J Pathol. 2004; 164:521-32).

Long-term feeding of dTGR (from week 4 to 7 of age) with a mixture of pure EPA- and DHA-ethyl esters (Omacor from Solvay Arzneimittel, Hannover, Germany) improved the electrical remodeling of the heart in this model of angiotensin II-induced hypertension. In particular, EPA and DHA reduced the mortality, suppressed the inducibility of cardiac arrhythmias and protected against connexin 43-gap junctional remodeling (Fischer et al., Hypertension. 2008 February; 51(2):540-6). In general, CYP-dependent eicosanoids have to be considered as second messengers: EETs and 20-HETE are produced by CYP enzymes after extracellular signal induced release of AA from membrane phospholipids (by phospholipase A2) and exert their function in the context of signaling pathways modulating ion transport, cell proliferation and inflammation. Depending on the diet, n-3 PUFAs partially replace AA at the sn2-position of phospholipids and may thus become involved as alternative molecules in the subsequent signaling pathways.

The few studies on the biological activities of CYP-dependent eicosanoids in the heart indicate important roles for EETs and 20-HETE in the regulation of L-type Ca²⁺ and sarcolemmal and mitochondrial ATP-sensitive potassium (KATP) channels. In cardiac myocytes, L-type Ca²⁺ currents and cell shorting are reduced upon inhibition of EET generation and these effects can be reversed by adding 11,12-EET (Xiao et al., J Physiol. 1998; 508 (Pt 3):777-92). EETs were also shown to activate cardiac KATP channels. This effect is highly stereoselective: only the S,R but not the R,S-enantiomer of 11,12-EET was effective (Lu et al., Mol Pharmacol. 2002; 62:1076-83). Overexpression of the EET-generating human CYP2J2 resulted in an improved postischemic functional recovery of the transgenic mouse heart via activation of KATP channels (Seubert et al., Circ Res. 2004; 95:506-14). 20-HETE appears to play an opposite role by acting as an endogenous KATP channel blocker (Gross et al., J Mol Cell Cardiol. 2004; 37:1245-9; Nithipatikom et al., Circ Res. 2004; 95:e65-71).

Although n-3 PUFA-derived CYP metabolites, such as 17,18-EEQ and 19,20-EDP, play important roles in mediating the beneficial effects of n-3 PUFAs in the mammalian body, they are not used as therapeutics due to their limited bioavailability as well as chemical and metabolic instability. These epoxymetabolites of n-3 PUFAs are prone to autoxidation, rapid inactivation by the soluble epoxide hydrolase, and degradation by β-oxidation. Improved analogues of n-3 PUFA metabolites have been disclosed in WO2017/013264 A1 that have significantly improved pharmacological properties compared to epoxymetabolites of n-3 PUFAs.

Growth differentiation factor-15 (GDF-15), also known as ‘macrophage inhibitory cytokine-1 (MIC-1), placental transformation growth factor (PTGF-b), prostate derived factor (PDF), placental bone morphogenetic protein (PLAB), prostate derived factor, or nonsteroidal anti-inflammatory drug activated gene-1 (NAG-1), is a marker of inflammation, oxidative stress and it is associated with adverse prognosis in cardiovascular disease. GDF-15 was shown to be associated with major adverse cardiovascular events (MACE) and all-cause death in patients with coronary artery disease (CAD). It is thus useful as a prognostic marker indicating long-term MACE and all-cause death (see Li et al., Cardiovasc Diabetol, 2020, 19:120; Daniels et al., Circulation, 2011, 123:2101-2110). Furthermore, studies identified GDF-15 to be associated with other diseases such as hypertension, diabetes, heart failure, renal functions and concentrations of N-terminal pro-B-type natriuretic peptide (NT-proBNP) (see Eggers et al., Clinical Chemistry 59:7, 2013). GDF-15 was further identified as a strong predictor for all-cause mortality and strongly associated with many functional parameters and key biomarkers, independently of age and sex (Rothenbacher et al., Age and ageing 2019, 48:541-546). In a further study that investigated biomarkers associated with cardiovascular death in patients suffering from atrial fibrillation (AF) GDF-15 was identified as being strongly associated with the underlying mechanisms of oxidative stress and inflammation (see Pol et al., Cardiovascular Research, 2021). In summary GDF-15 is a factor closely associated with cardiac diseases, cardiovascular disease, hypertension, diabetes and renal function and is thus of significant importance as prognostic marker for these diseases, but may also be involved as a factor involved in the mechanisms underlying these diseases. Recent publications have suggested that GDF-15 might also be relevant as a therapeutic target for cardiovascular diseases (Rochette et al., Int. J.Mol. Sci. 2021, 22, 8889; WO 2015/054399 A1), cancer and metabolic diseases (Wischhusen et al., 2020, Front. Immunol. 11:951). Any therapeutic approaches that can decrease circulating GDF-15 levels may thus be relevant for the treatment of diseases associated with circulating GDF-15.

The present invention provides first experimental data that GDF-15 and other important biomarkers are lowered by improved analogues of n-3 PUFA metabolites. The lowered GDF-15 levels furthermore resulted in a therapeutic improvement in these patients indicating that the improved analogues of n-3 PUFA metabolites of the present invention are capable of treating diseases associated with elevated GDF-15 levels.

In a first aspect the above problem is solved by the provision of compounds of the general formula (I):

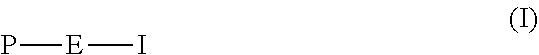

or a pharmaceutically acceptable salt thereof, wherein
P is a group represented by the general formula (II):

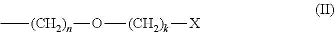

wherein
n is 0 or an integer of from 3 to 8, i.e. 3, 4, 5, 6, 7, or 8, preferably 3; and
k is 0, 1, or 2; preferably with the proviso that when n is 0 k is 1, most preferably k is 1;
X represents CH₂OH, CH₂OAc, CH(O) or a group selected from the group consisting of:

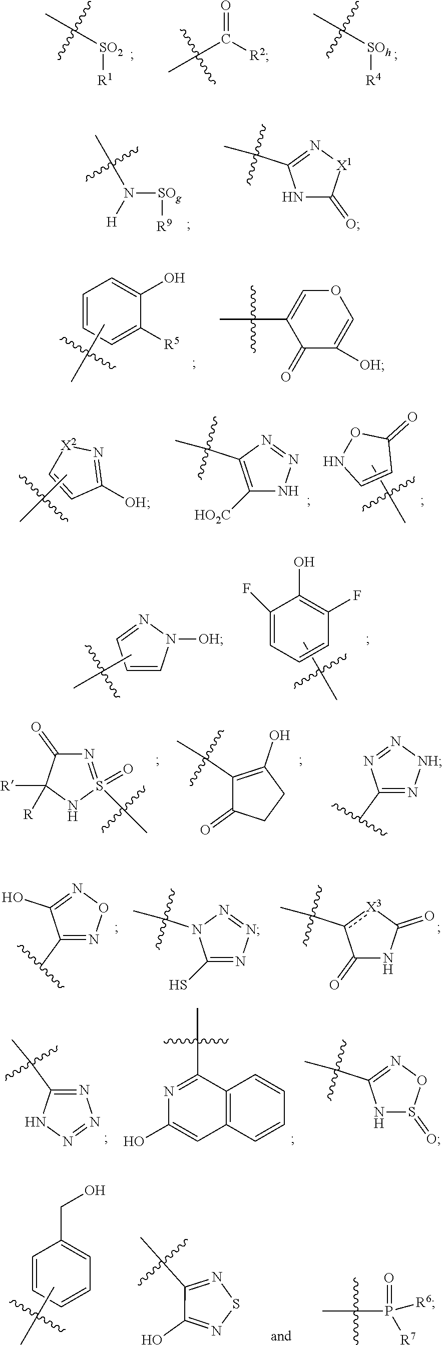

preferably X is

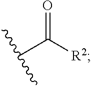

wherein
R and R′ each independently represents a hydrogen atom; or a C₁-C₆alkyl group which may be substituted with one or more fluorine or chlorine atom(s) or hydroxyl group(s);
R¹ represents a hydroxyl group, C₁-C₆alkoxy, NHCN, NH(C₁-C₆alkyl), —NH(C₃-C₆cycloalkyl), —NH(aryl), or O(C₁-C₆alkyldiyl)O(C═O)R¹¹; R¹¹ is a C₁-C₆alkyl group which is optionally substituted with one or more fluorine or chlorine atom(s); or a C₃-C₆cycloalkyl group which is optionally substituted with one or more fluorine or chlorine atom(s) or hydroxyl group(s);
R² represents —NHR³; —NR²⁰R²¹; —OR²²; —(OCH₂—CH₂)ᵢ—R²³; —C₃-C₁₀-heterocyclyl optionally substituted with one, two or three substituents independently selected from the group consisting of hydroxyl group, C₁-C₆alkoxy, C₁-C₆alkyl, and oxo; -(Xaa)ₒ; a mono-, or disaccharide, or a derivative thereof, which is joined to —C(O) by an ester bond via the 1-O—, 3-O—, or 6-O-position of the saccharide; or is selected from the group consisting of:

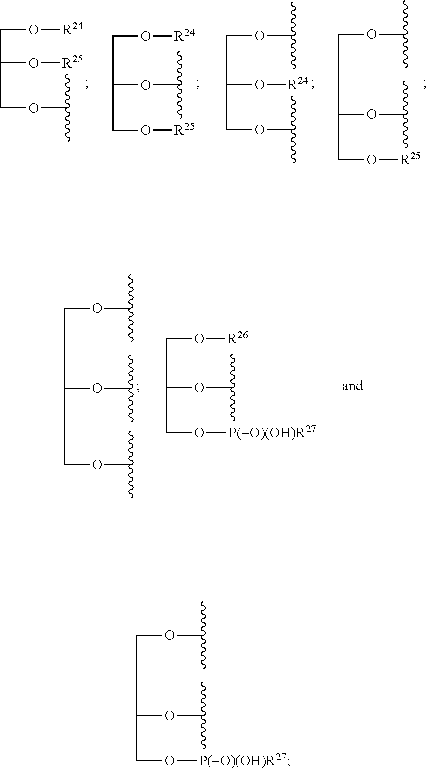

wherein
R³ represents (SO₂R³⁰); (OR³¹); —C₁-C₆alkanediyl(SO₂R³²); —C₁-C₆alkanediyl(CO₂H), an aryl group, a heteroaryl group, a cycloalkyl group or a heterocycloalkyl group, wherein the aryl group is optionally substituted with one, two or three substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), N(C₁-C₆)dialkyl, and —C(═O)OR⁵¹; wherein the heteroaryl group, is optionally substituted with one, two or three substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), N(C₁-C₆)dialkyl and —C(═O)OR⁵¹; where the cycloalkyl group is optionally substituted with one, two or three substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), N(C₁-C₆)dialkyl, and —C(═O)OR⁵¹; and wherein the heterocycloalkyl group is optionally substituted with one, two or three substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), —N(C₁-C₆)dialkyl and —C(═O)OR⁵¹;
R³⁰ is a C₁-C₆alkyl, or an aryl group, wherein the C₁-C₆alkyl group is optionally substituted with —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, one, two or three fluorine or chlorine atoms, or a hydroxyl group; and wherein the aryl group is optionally substituted with one, two or three substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), and N(C₁-C₆)dialkyl;
R³¹ is a C₁-C₆alkyl group which is optionally substituted with one or more fluorine or chlorine atom(s) or hydroxyl group(s); or a C₃-C₆cycloalkyl group which is optionally substituted with one or more fluorine or chlorine atom(s) or hydroxyl group(s);
R³² is a C₁-C₆alkyl group which is optionally substituted with one or more fluorine or chlorine atom(s) or hydroxyl group(s); or a C₃-C₆cycloalkyl group which is optionally substituted with one or more fluorine or chlorine atom(s) or hydroxyl group(s);
R²⁰ and R²¹ each independently represents a hydrogen atom; a C₁-C₆alkyl group which may be substituted with one or more fluorine or chlorine atom(s) or hydroxyl group(s); a C₃-C₆cycloalkyl group which may be substituted with one or more fluorine or chlorine atom(s) or hydroxyl group(s); —C₁-C₆alkyldiyl(CO₂H) or together form a C₃-C₁₀-heterocycloalkyl which may be substituted with one or more C₁-C₆alkyl group(s), C₁-C₆alkoxy group(s), fluorine or chlorine atom(s) or hydroxyl group(s);
R²² is a hydrogen atom, a C₁-C₆alkyl group; or a C₃-C₆cycloalkyl group; wherein the C₁-C₆alkyl group or the C₃-C₆cycloalkyl group is optionally substituted with —NH₂, —NH(C₁-C₆)alkyl, N(C₁-C₆)dialkyl, NH(C₁-C₆)alkyldiyl- C₁-C₆alkoxy, one, two or three fluorine or chlorine atom(s), hydroxyl, or C₁-C₆alkoxy, an aralkyl group, a heteroalkyl group or a heteroalkylcycloalkyl group;
R²³ is —OH, —O(C₁-C₃)alkyl, or —N(C₁-C₃)dialkyl;
i is an integer of from 1 to 10;
R²⁴, R²⁵, and R²⁶ each independently represents a hydrogen atom; C(═O)C₁₁-C₂₁alkyl; or C(═O)C₁₁-C₂₁alkenyl;
R²⁷ represents OH; O(CH₂)₂NH₂, OCH₂—[CH(NH₂)(CO₂H)], O(CH₂)₂N(CH₃)₃; or

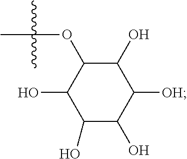 [SMILES: *OC1C(O)C(O)C(O)C(O)C1O]

Xaa represents Gly, a conventional D,L-, D- or L-amino acid, a non-conventional D,L-, D- or L-amino acid, or a 2- to 10-mer peptide; and is joined to —C(═O) by an amide bond;
o is an integer of from 1 to 10;
R⁴ is selected from the group consisting of:

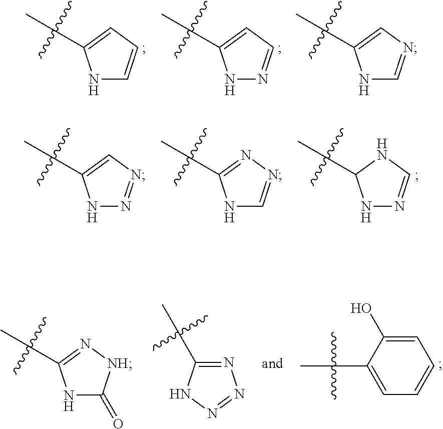

h is 0, 1, or 2;
R⁵ represents a hydrogen atom; a fluorine or chlorine atom; —CF₃; —C(═O)OR⁵¹;
—NHC(═O)R⁵²; —C(═O)NR⁵³R⁵⁴; or —S(O₂)OH;
R⁵¹ represents a hydrogen atom; a C₁-C₆alkyl group; or a C₃-C₆cycloalkyl group; wherein the C₁-C₆alkyl group or the C₃-C₆cycloalkyl group is optionally substituted with —NH₂, —NH(C₁-C₆)alkyl, N(C₁-C₆)dialkyl, NH(C₁-C₆)alkyldiyl-C₁-C₆alkoxy, one, two or three fluorine or chlorine atom(s), hydroxyl, or C₁-C₆alkoxy;
R⁵², R⁵³ and R⁵⁴ each independently represents a C₁-C₆alkyl group which is optionally substituted with one or more fluorine or chlorine atom(s); a C₃-C₆cycloalkyl group which is optionally substituted with one or more fluorine or chlorine atom(s); or an aryl group which is optionally substituted with one, two or three substituents independently selected from the group consisting of C₁-C₆alkyl, C₁-C₆haloalkyl, C₁-C₆alkoxy, C₁-C₆alkylthio, fluorine or chlorine atom, hydroxyl group, amino group, NH(C₁-C₆alkyl), N(C₁-C₆)dialkyl, and an oxo substituent;
R⁶ and R⁷ each independently represents a hydroxyl group; an —O(C₁-C₆)alkyl group, an —O(C₂-C₆)alkenyl group, a, —O(C₁-C₆)alkyldiylO(C═O)(C₁-C₆)alkyl group, or a —O(C₁-C₆)alkyldi-ylO(C=O)(C₂-C₆)alkenyl group; wherein the C₁-C₆alkyl group and the C₂-C₆alkenyl group may be substituted with NH₂, —NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆ alkylaminocarbonyl-, di(C₁-C₆)alkylamino-carbonyl-, or one, two or three fluorine or chlorine atom(s); or
R⁶ represents a hydroxyl group and R⁷ represents a group:

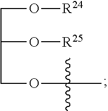

R⁹ represents C₁-C₆alkyl, or aryl; wherein the C₁-C₆alkyl is optionally substituted with —NH₂, NH(C₁-C₆)alkyl, N(C₁-C₆)dialkyl, NH(C₁-C₆)alkyldiyl-C₁-C₆alkoxy, one, two or three fluorine or chlorine atom(s), hydroxy, C₁-C₆alkoxy, aryl, aryloxy, C(═O)-aryl, C(═O)C₁-C₆alkoxy; and wherein the aryl group is optionally substituted with one, two or three substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), N(C₁-C₆)dialkyl, and an oxo substituent;
g is 1 or 2, preferably 2;
X¹ represents an oxygen atom; sulfur atom; or NH;
X² represents an oxygen atom; sulfur atom; NH; or N(CH₃);
X³ represents an oxygen atom; sulfur atom; nitrogen atom; carbon atom; or C—OH; and the dashed line represents a carbon-carbon bond or a carbon-carbon double bond;
E is a group represented by the general formula (III) or (IV):

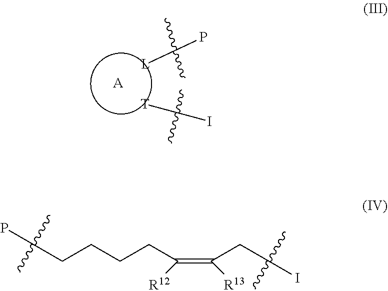

wherein R¹² and R¹³ are preferably in cis configuration, and wherein
ring A in formula (III) represents a 5-membered or 6-membered carbocyclic or heterocyclic ring containing at least one double bond, including an aromatic carbocyclic or heterocyclic ring, which can be substituted with one to three or one to four substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), and N(C₁-C₆)dialkyl; and L and T each independently represents a ring atom, wherein L and T are adjacent to another;
R¹² and R¹³ each independently represents a hydrogen atom, a fluorine atom, hydroxyl, —NH₂, C₁-C₆alkyl, C₁-C₆alkoxy, C(═O)-aryl, C(═O)C₁-C₆alkyl, or —SO₂(C₁-C₆alkyl); or —SO₂aryl; wherein any of the foregoing C₁-C₆alkyl, C₁-C₆alkoxy, or aryl are optionally substituted with one, two or three substituents independently selected from the group consisting of —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, fluorine or chlorine atom, and hydroxyl; or R² and R¹³ are taken together to form a 5-membered or 6-membered ring, which ring is optionally substituted with one, two or three substituents independently selected from the group consisting of —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, fluorine or chlorine atom, and hydroxyl;
I is —(CH₂)m-Y, wherein
m is an integer of from 3 to 6, i.e 3, 4, 5, or 6, provided that m is an integer of from 3 to 5 when E is a group according to general formula (III);
Y represents —U—V-W-(CH₂)ₚ—(CH₃)_{q}, wherein p is an integer from 0 to 6; q is 0 or 1; U is absent or selected from the group consisting of CH, CH₂ and NR⁴⁰, with the proviso that U is only CH if it forms an epoxy group together with V and W; V is selected from the group consisting of —C(O)—, —C(O)—C(O)—, —O—, and —S—; W is selected from the group consisting of CH, CH₂ and NR⁴⁰ with the proviso that W is only CH if it forms an epoxy group together with U and V;
or Y represents a group selected from the group consisting of:

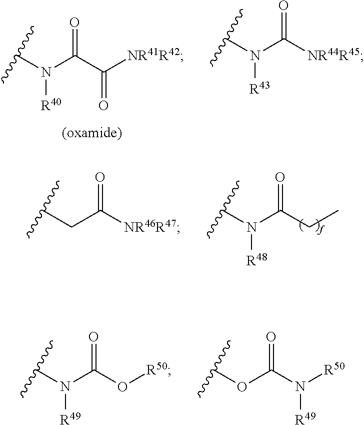

wherein
R⁴⁰, R⁴¹, R⁴³, R⁴⁴, R⁴⁶, R⁴⁸ and R⁴⁹ each independently represents a hydrogen atom, —C₁-C₆alkyl, —C₃-C₆cycloalkyl, —C₁-C₆alkoxy, C(═O)aryl, or C(═O)C₁-C₆alkyl, wherein any of the foregoing C₁-C₆alkyl, C₃-C₆cycloalkyl, C₁-C₆alkoxy, or aryl are optionally substituted with one, two or three substituents independently selected from the group consisting of —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, fluorine or chlorine atom, and hydroxy; or R⁴⁰ and R⁴¹, or R⁴³ and R⁴⁴, are taken together to form a 5-membered or 6-membered ring, which ring may be substituted with one, two or three substituents independently selected from the group consisting of —NH₂, —NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, fluorine or chlorine atom, and hydroxyl;
R⁴², R⁴⁵, R⁴⁷ and R⁵⁰ each independently represents a —C₁-C₃alkyl, wherein the C₁-C₃alkyl may be substituted with one, two or three substituents independently selected from the group consisting of —NH₂, NH(C₁-C₃)alkyl, N(C₁-C₃)dialkyl, C₁-C₃alkylcarbonyloxy-, C₁-C₃alkoxycarbonyloxy-, C₁-C₃alkylcarbonylthio-, C₁-C₃alkylaminocarbonyl-, di(C₁-C₃)alkylaminocarbonyl-, fluorine or chlorine atom, and hydroxyl; or R⁴⁰ and R⁴¹; R⁴³ and R⁴⁴; R⁴⁹ and R⁵⁰ are taken together to form a 5-membered or 6-membered ring, which ring may be substituted with one, two or three substituents independently selected from the group consisting of —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, fluorine or chlorine atom, and hydroxyl;
f is an integer of from 0 to 2;
with the proviso that
when X does not comprise a —C(═O)O-motif with the carbonyl carbon in alpha or beta position to the oxygen atom of general formula (II), Y is an oxamide, a carbamate or a carbamide, preferably Y is an oxamide as defined above
for use in treating, reducing the risk of developing or preventing a disorder associated with an elevated GDF-15 plasma concentration, preferably wherein the GDF-15 plasma concentration is at least 1000 ng/L, preferably wherein the disease associated with an elevated GDF-15 plasma concentration is selected from a cardiovascular disease and a metabolic disease.

In a preferred embodiment, wherein the disorder associated with an elevated GDF-15 plasma concentration is a metabolic disease, preferably diabetes mellitus, more preferably type 2 diabetes, most preferably pre-Diabetes, the GDF-15 plasma concentration is at least 500 ng/L.

In a preferred embodiment, the compounds of present invention are compounds of formula (I) as described above with the proviso that
when X does not comprise a —C(═O)O-motif with the carbonyl carbon in alpha or beta position to the oxygen atom of general formula (II), Y is an oxamide, a carbamate or a carbamide, preferably Y is an oxamide as defined above.

In a preferred embodiment, the compounds of formula (I) are compounds as described above with the further proviso that
when n is 3, 5, 6, 7 or 8, preferably 3 k is 1 and E is a group according to general formula (III) or general formula (IV), wherein each of R¹² and R¹³ is a hydrogen atom;
P represents a group:

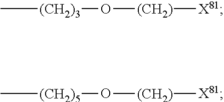

wherein
X⁸¹ represents a group selected from the group consisting of:

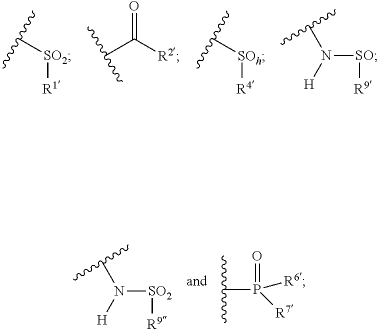

R^{1′} is defined as R¹ above;
R^{2′} represents —NHR^{3′}; —OR^{22′}; —(OCH₂—CH₂)ᵢ—R²³; a mono-, or disaccharide, or a derivative thereof, which is joined to —C(═O) by an ester bond via the 1-O—, 3-O—, or 6-O-position of the saccharide;
or wherein R² is selected from the group consisting of:

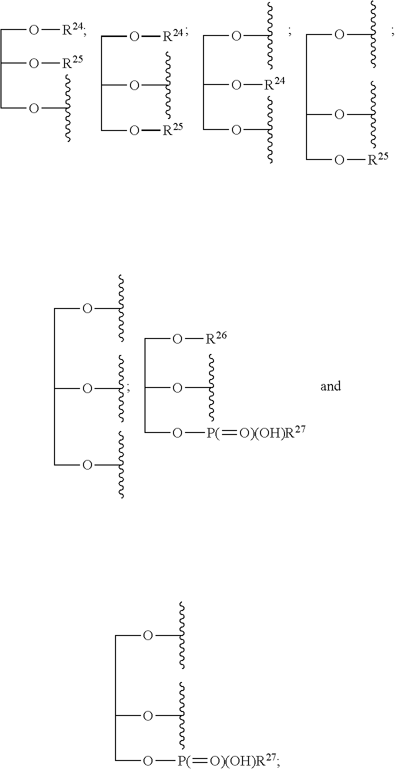

wherein
R^{3′} represents (SO₂R³⁰); (OR³¹); —C₁-C₆alkanediyl(SO₂R³²); or —C₂-C₆alkanediyl(CO₂H);
R^{22′} is a hydrogen or a C₃-C₆cycloalkyl group, which is optionally substituted with —NH₂, NH(C₁-C₆)alkyl, N(C₁-C₆)dialkyl, NH(C₁-C₆)alkyldiyl- C₁-C₆alkoxy, one, two or three fluorine or chlorine atom(s), hydroxy, or C₁-C₆alkoxy;
R²³ and i are as defined above;
R²⁴, R²⁵, R²⁶, and R²⁷ are as defined above;
R^{4′} is defined as R⁴ above; and h is defined as above;
R^{6′} and R^{7′} are defined as R⁶ and R⁷ above;
R^{9′} is defined as R⁹ above; R^{9′} represents aryl which is optionally substituted with one, two or three substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), N(C₁-C₆)dialkyl, and an oxo substituent.

In a more preferred embodiment the compound of the present invention is one, wherein X is

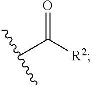

wherein R² is —OR²²; —(OCH₂—CH₂)ᵢ—R²³; a mono-, or disaccharide, or a derivative thereof, which is joined to —C(═O) by an ester bond via the 1-O—, 3-O—, or 6-O-position of the saccharide;
or wherein R² is selected from the group consisting of:

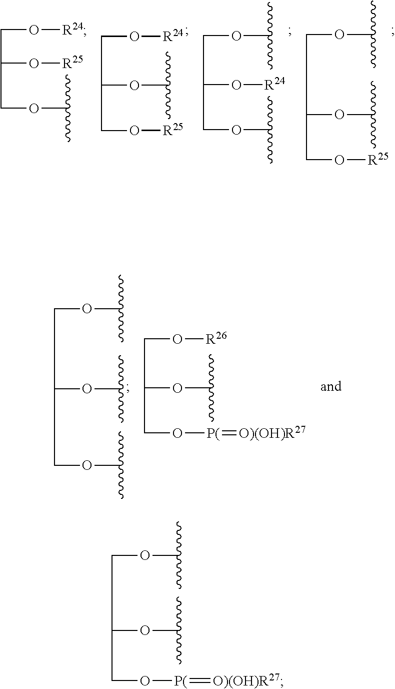

wherein R²³ and i are as defined above, preferable i is 3;
and wherein R²², and R²³ to R²⁷ are as defined in claim 1, preferably R²² is a hydrogen atom or a C₁-C₆alkyl group, more preferably a hydrogen atom.

In a further more preferred embodiment, the compound of the present invention is one, wherein X is —C(═O)OH or a suitable salt of the carboxylic acid, preferably a free carboxylic acid.

In another more preferred embodiment, the compound of the present invention is one, wherein Y is one of the oxamides as defined above.

It is further preferred that the compound of the present invention is one, wherein X is

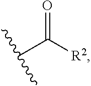

wherein R² is —OR²²; —(OCH₂—CH₂)ᵢ—R²³; a mono-, or disaccharide, or a derivative thereof, which is joined to —C(═O) by an ester bond via the 1-O—, 3-O—, or 6-O-position of the saccharide; or wherein R² is selected from the group consisting of:

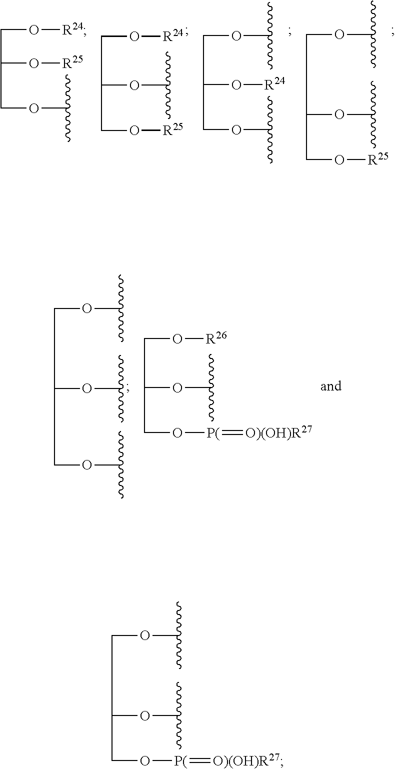

wherein and R²², R²³ to R²⁷ and i are as defined above, preferably R²² is a hydrogen atom or a C₁-C₆alkyl group, more preferably a hydrogen atom, preferably i is 2 to 4, more preferably 3, and wherein Y is preferably one of the oxamides defined above.

In a more preferred embodiment, the compound of the present invention is one, wherein X is C(═O)OH, preferably the free carboxylic acid, and Y is preferably one of the oxamides defined above.

In another more preferred embodiment, the compound of the present invention is one with the following formula (V):

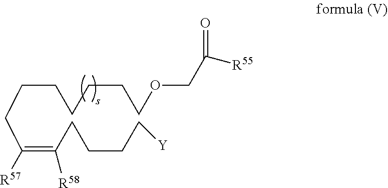

wherein
R⁵⁵ represents —OH; —OR²²; —(OCH₂—CH₂)i-R²³; a mono-, or disaccharide, or a derivative thereof, which is joined to —C(═O) by an ester bond via the 1-O—, 3-O—, or 6-O-position of the saccharide;
R²², R²³ and i are as defined above, preferably R²² is a hydrogen atom or a C₁-C₆alkyl group, more preferably a hydrogen atom and i is preferably 2 to 4, more preferably 3;
Y represents a group selected from the group consisting of:

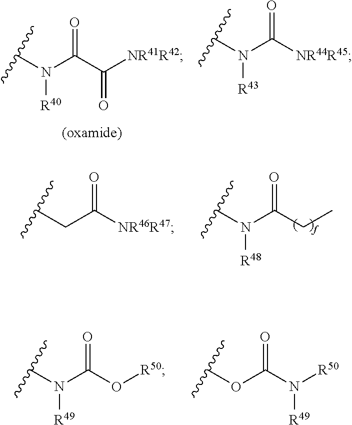

wherein

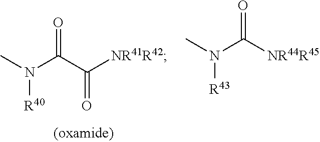

are preferred, and

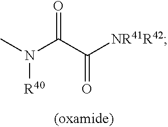

is particularly preferred; and
wherein R⁴⁰ to R⁵⁰ are defined above, preferably R⁴⁰ is a hydrogen atom or a C₁-C₆alkyl group, more preferably a hydrogen atom
R⁵⁷ and R⁵⁸ are hydrogen; or form together a five- or six-membered ring, preferably an aromatic ring, optionally substituted with one to three or one to four substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), N(C₁-C₆)dialkyl, and an oxo substituent;
s is 0, 1 or 2, with the proviso that s is 0 if R⁵⁷ and R⁵⁸ form together a five- or six-membered ring;
the double bond in formula (V) represents a double carbon-carbon bond in cis- configuration, if R⁵⁷ and R⁵⁸ are hydrogen, or this double bond is part of a five- or six-membered ring formed together by R⁵⁷ and R⁵⁸.

In a further most preferred embodiment the compounds of formula (V) are those wherein
R⁵⁵ represents —OH or —(OCH₂—CH₂)ᵢ—R²³; i is 2 to 4, preferably i is 3; R²³ is preferably OH;
Y is an oxamide, a carbamide or a carbamate, preferably a C₁-C₆alkyl substituted oxamide, carbamide or carbamate;
R⁵⁷ and R⁵⁸ are both H, or together form a substituted or non-substituted five- or six-membered aromatic ring, preferably form a substituted or non-substituted benzyl ring; and
s is 1 or s is 0 if R⁵⁷ and R⁵⁸ together form a substituted or non-substituted five- or six-membered aromatic ring.

The most preferred specific compounds of the present invention are those selected from the group consisting of:

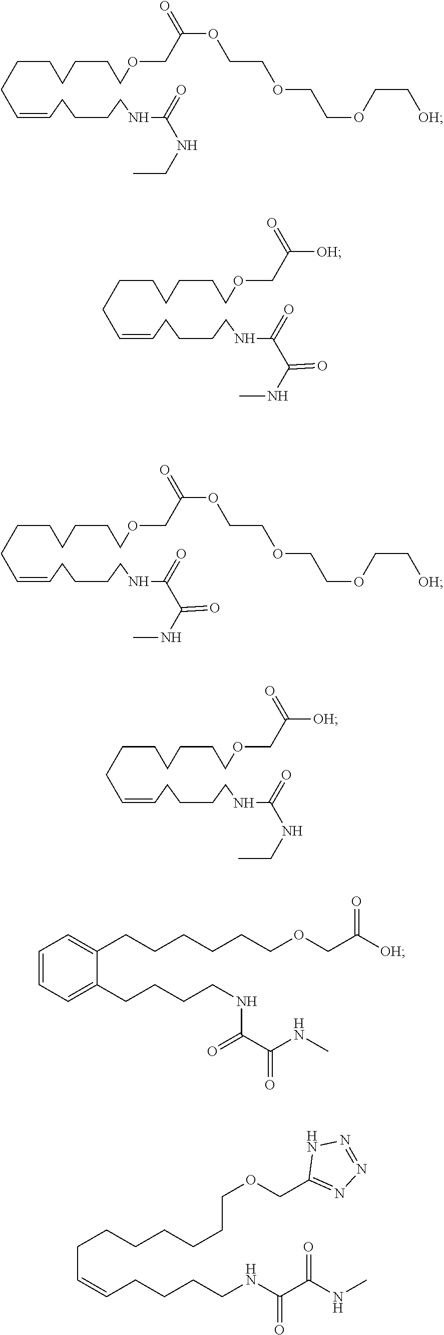 [SMILES: CCNC(=O)NCCCC/C=C\CCCCCCCOCC(=O)OCCOCCOCCO ; CNC(=O)C(=O)NCCCC/C=C\CCCCCCCOCC(=O)O ; CNC(=O)C(=O)NCCCC/C=C\CCCCCCCOCC(=O)OCCOCCOCCO ; CCNC(=O)NCCCC/C=C\CCCCCCCOCC(=O)O ; CNC(=O)C(=O)NCCCCc1ccccc1CCCCCCOCC(=O)O ; CNC(=O)C(=O)NCCCC/C=C\CCCCCCCOCc1nnn[nH]1]

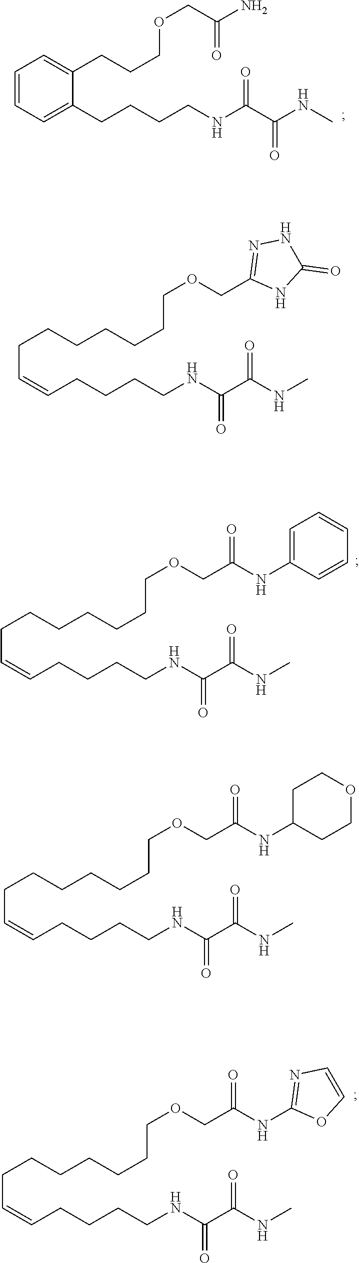 [SMILES: CNC(=O)C(=O)NCCCCc1ccccc1CCCOCC(N)=O ; CNC(=O)C(=O)NCCCC/C=C\CCCCCCCOCc1n[nH]c(=O)[nH]1 ; CNC(=O)C(=O)NCCCC/C=C\CCCCCCCOCC(=O)Nc1ccccc1 ; CNC(=O)C(=O)NCCCC/C=C\CCCCCCCOCC(=O)NC1CCOCC1 ; CNC(=O)C(=O)NCCCC/C=C\CCCCCCCOCC(=O)Nc1ncco1]

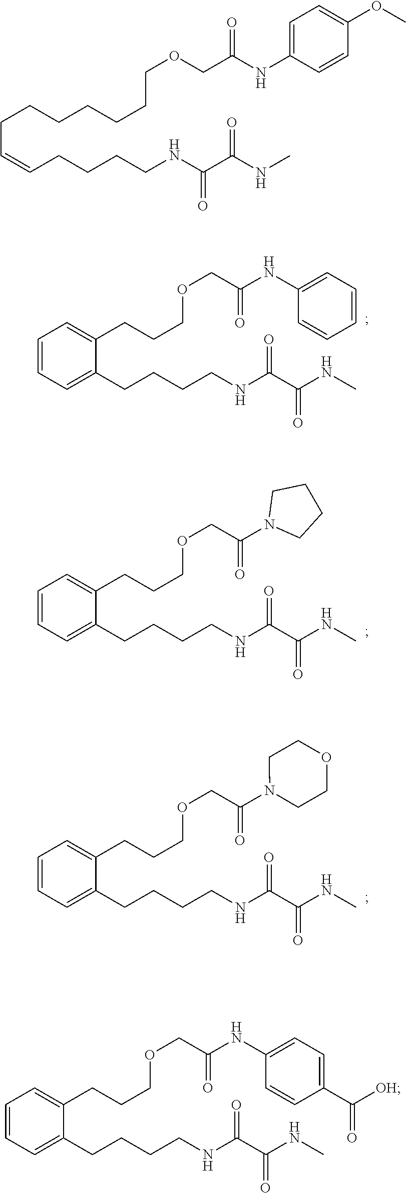 [SMILES: CNC(=O)C(=O)NCCCC/C=C\CCCCCCCOCC(=O)Nc1ccc(OC)cc1 ; CNC(=O)C(=O)NCCCCc1ccccc1CCCOCC(=O)Nc1ccccc1 ; CNC(=O)C(=O)NCCCCc1ccccc1CCCOCC(=O)N1CCCC1 ; CNC(=O)C(=O)NCCCCc1ccccc1CCCOCC(=O)N1CCOCC1 ; CNC(=O)C(=O)NCCCCc1ccccc1CCCOCC(=O)Nc1ccc(C(=O)O)cc1]

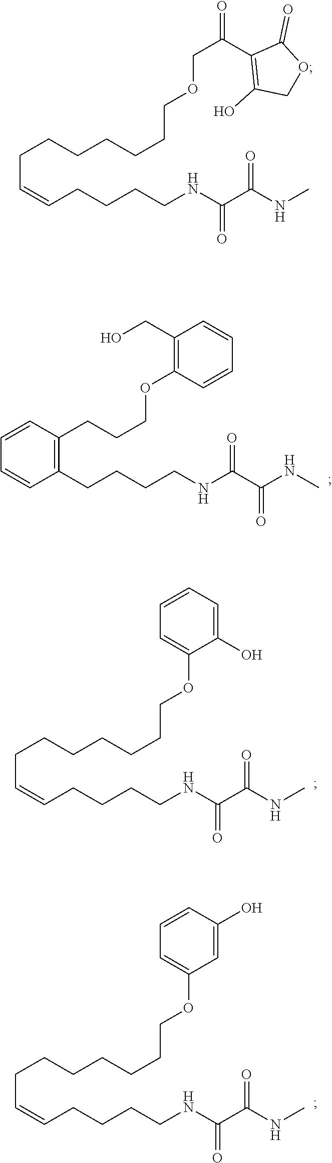 [SMILES: CNC(=O)C(=O)NCCCC/C=C\CCCCCCCOCC(=O)C1=C(O)COC1=O ; CNC(=O)C(=O)NCCCCc1ccccc1CCCOc1ccccc1CO ; CNC(=O)C(=O)NCCCC/C=C\CCCCCCCOc1ccccc1O ; CNC(=O)C(=O)NCCCC/C=C\CCCCCCCOc1cccc(O)c1]

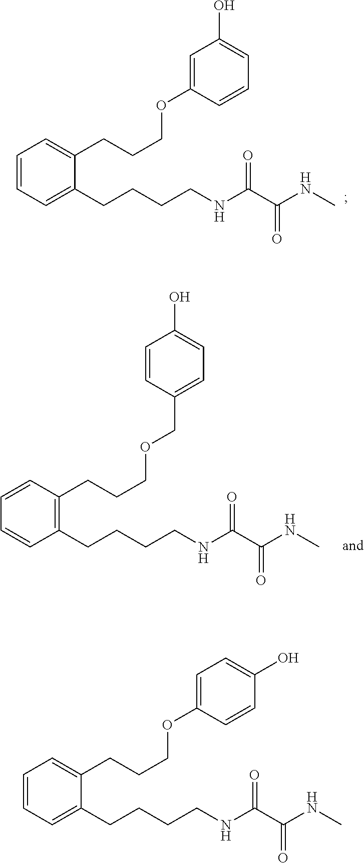 [SMILES: CNC(=O)C(=O)NCCCCc1ccccc1CCCOc1cccc(O)c1 ; CNC(=O)C(=O)NCCCCc1ccccc1CCCOCc1ccc(O)cc1 ; CNC(=O)C(=O)NCCCCc1ccccc1CCCOc1ccc(O)cc1]

or a pharmaceutically acceptable salt thereof.

Among the above, the compound with the following formula (VI)

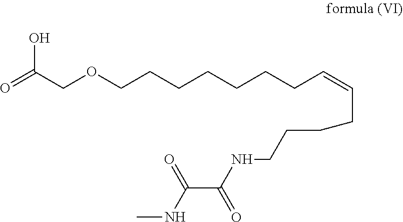 [SMILES: CNC(=O)C(=O)NCCCC/C=C\CCCCCCCOCC(=O)O]

or a pharmaceutically acceptable salt thereof is most preferred.

In one embodiment the compound is of the general formula (I):

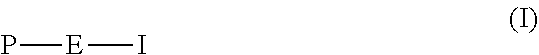

or a pharmaceutically acceptable salt thereof, wherein
P is a group represented by the general formula (II):

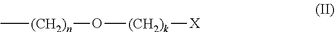

wherein
n is 0 or an integer of from 3 to 8; and
k is 0 or 1; preferably with the proviso that when n is 0 k is 1, most preferably k is 1;
X represents CH₂OH, CH₂OAc, CH(O) or a group selected from the group consisting of:

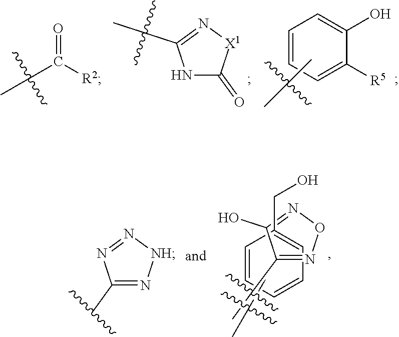

and/preferably X is

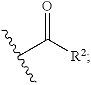

wherein R² represents —NHR³; —NR²⁰R²¹; —OR²²; —(OCH₂—CH₂)ᵢ—R²³; —C₃-C₁₀-heterocyclyl optionally substituted with one, two or three substituents independently selected from the group consisting of hydroxyl group, C₁-C₆alkoxy, C₁-C₆alkyl, and oxo;
wherein R³ represents a phenyl group, a 5-membered heteroaryl group with 1 to 4 ring heteroatoms selected from nitrogen, oxygen, and sulphur or a 5-6-membered heterocycloalkyl group with 1 to 2 ring heteroatoms selected from nitrogen, oxygen, or sulphur, wherein the phenyl group is optionally substituted with one, two or three substituents independently selected from the group consisting of C₁-C₆alkyl, C₁-C₆alkoxy and —C(═O)OR⁵¹;
R²⁰ and R²¹ each independently represents a hydrogen atom; a C₁-C₆alkyl group which may be substituted with one or more hydroxyl group(s); a C₃-C₆cycloalkyl group or together form a C₃-C₁₀-heterocycloalkyl;
R²² is a hydrogen atom, a C₁-C₆alkyl group;
R²³ is —OH;
i is an integer of from 1 to 10;
R⁵ represents a hydrogen atom; a fluorine or chlorine atom; —CF₃; —C(═O)OR⁵¹;
—NHC(═O)R⁵²; —C(═O)NR⁵³R⁵⁴; or —S(O₂)OH;
R⁵¹ represents a hydrogen atom; a C₁-C₆alkyl group;
R⁵², R⁵³ and R⁵⁴ each independently represents a C₁-C₆alkyl group;
X¹ represents an oxygen atom; sulfur atom; or NH;
E is a group represented by the general formula (III) or (IV):

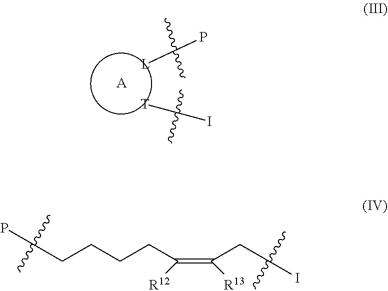

wherein R¹² and R¹³ are preferably in cis configuration, and wherein
ring A in formula (III) represents a 5-membered or 6-membered carbocyclic ring containing at least one double bond, including an aromatic carbocyclic ring; and L and T each independently represents a ring atom, wherein L and T are adjacent to another;
R¹² and R¹³ each independently represents a hydrogen atom, a fluorine atom, hydroxyl;
I is —(CH₂)ₘ—Y, wherein
m is an integer of from 3 to 6, provided that m is an integer of from 3 to 5 when E is a group according to general formula (III);
Y represents —U—V-W-(CH₂)ₚ—(CH₃)_{q}, wherein p is an integer from 0 to 6; q is 0 or 1; U is absent or selected from the group consisting of CH, CH₂ and NR⁴⁰, with the proviso that U is only CH if it forms an epoxy group together with V and W; V is selected from the group consisting of —C(O)—, —C(O)—C(O)—, —O—, and —S—; W is selected from the group consisting of CH, CH₂ and NR⁴⁰ with the proviso that W is only CH if it forms an epoxy group together with U and V;
or Y represents a group selected from the group consisting of:

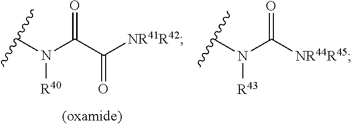

wherein
R⁴⁰, R⁴¹, R⁴³, R⁴⁴ each independently represents a hydrogen atom, —C₁-C₆alkyl;
R⁴², R⁴⁵ each independently represents a —C₁-C₃alkyl
with the proviso that
when X does not comprise a —C(═O)O-motif with the carbonyl carbon in alpha or beta position to the oxygen atom of general formula (II), Y is an oxamide, a carbamate or a carbamide, preferably Y is an oxamide as defined above.

In a preferred embodiment, the compounds of formula (I) are compounds as described above with the further proviso that
when n is 3 or 5, k is 1 and E is a group according to general formula (III) or general formula (IV), wherein each of R¹² and R¹³ is a hydrogen atom;
P represents a group:

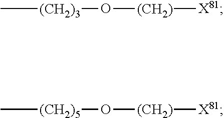

wherein
X⁸¹ represents a group selected from the group consisting of:

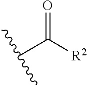

R^{2′} represents —NHR^{3′}; —OR^{22′}; —(OCH₂—CH₂)ᵢ—R²³;
wherein
R^{3′} represents C₆-aryl;
R^{22′} is a hydrogen;
R²³ and i are as defined above;

It is further preferred that the compound of the present invention is one, wherein X is

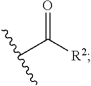

wherein R² is —OR²²; —(OCH₂—CH₂)ᵢ—R²³;
wherein R²³ and i are as defined above;
and wherein R²², and R²³ are as defined in claim 1.

It is further preferred that the compound of the present invention is one, wherein wherein X is —C(═O)OH or a suitable salt of the carboxylic acid, preferably a free carboxylic acid.

It is further preferred that the compound of the present invention is one, wherein X is

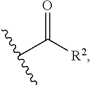

wherein R² is —OR²²; —(OCH₂—CH₂)ᵢ—R²³;
wherein and R²², R²³ and i are as defined in claim 1, and wherein Y is one of the oxamides defined according to claim 1.

It is further preferred that the compound of the present invention is one, wherein X is C(═O)OH, preferably the free carboxylic acid, and Y is one of the oxamides defined above.

In another more preferred embodiment, the compound of the present invention is one with the following formula (V):

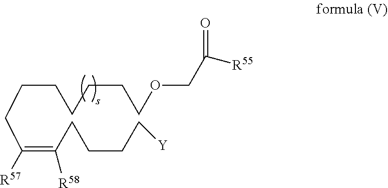

wherein
R⁵⁵ represents —OH; —OR²²; —(OCH₂—CH₂)ᵢ—R²³;
R²², R²³ and i are as defined in claim 1, preferably R²² is a hydrogen atom or a C₁-C₆alkyl group, more preferably a hydrogen atom and i is preferably 2 to 4, more preferably 3; Y represents a group selected from the group consisting of:

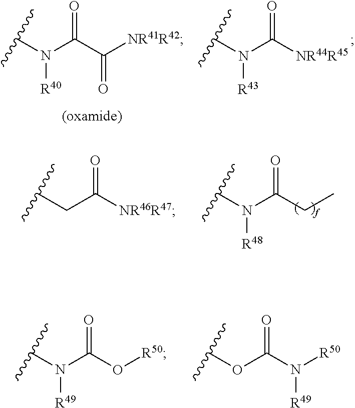

wherein R⁴⁰ to R⁵⁰ are defined in claim 1, preferably R⁴⁰ is a hydrogen atom or a C₁-C₆alkyl group, more preferably a hydrogen atom
R⁵⁷ and R⁵⁸ are hydrogen;
s is 0, 1 or 2;
the double bond in formula (V) represents a double carbon-carbon bond in cis- configuration, if R⁵⁷ and R⁵⁸ are hydrogen,.

In a further most preferred embodiment the compounds of formula (V) are those wherein R⁵⁵ represents —OH or —(OCH₂—CH₂)ᵢ—R²³; i is 2 to 4, preferably i is 3; R²³ is preferably OH;
Y is an oxamide, a carbamide or a carbamate, preferably a C₁-C₆alkyl substituted oxamide, carbamide or carbamate;
R⁵⁷ and R⁵⁸ are both H.

The compounds of the present invention have the advantage as demonstrated below in the experimental section that they are effective for treating, reducing the risk of developing or preventing a disorder associated with elevated GDF-15 plasma concentration, preferably a cardiovascular disease, or a metabolic disease. The elevated GDF-15 plasma concentration in these diseases is preferably at least 500 ng/L, 750 ng/L, 900 ng/L, 1000 ng/L, 1200 ng/L or 1500 ng/L, preferably at least 900 ng/L, 1000 ng/L, 1200 ng/L, more preferably at least 1000 ng/L. The compounds of the present invention are at the same time metabolically robust for pharmaceutical formulation and administration to subjects in need thereof.

The compounds described herein are generally described using standard nomenclature. For compounds having asymmetric centers, it is understood that, unless otherwise specified, all of the optical isomers and mixtures thereof are encompassed. Compounds with two or more asymmetric elements can also be present as mixtures of diastereomers. In addition, compounds with carbon-carbon double bonds may occur in Z- and E- forms, with all isomeric forms of the compounds being included in the present invention unless otherwise specified. Where a compound exists in various tautomeric forms, a recited compound is not limited to any one specific tautomer, but rather is intended to encompass all tautomeric forms. Recited compounds are further intended to encompass compounds in which one or more atoms are replaced with an isotope, i.e., an atom having the same atomic number but a different mass number. By way of general example, and without limitation, isotopes of hydrogen include tritium and deuterium and isotopes of carbon include ¹¹C, ¹³C, and ¹⁴C.

Compounds according to the formulas provided herein, which have one or more stereogenic center(s), have an enantiomeric excess of at least 50%. For example, such compounds may have an enantiomeric excess of at least 60%, 70%, 80%, 85%, 90%, 95%, or 98%. Some embodiments of the compounds have an enantiomeric excess of at least 99%. It will be apparent that single enantiomers (optically active forms) can be obtained by asymmetric synthesis, synthesis from optically pure precursors, biosynthesis, e.g. using modified CYP102 (CYP BM-3) or by resolution of the racemates, e.g. enzymatic resolution or resolution by conventional methods such as crystallization in the presence of a resolving agent, or chromatography, using, for example, a chiral HPLC column.

Certain compounds are described herein using a general formula that includes variables such as, e.g. P, E, I, R¹—R⁵⁰, X-X⁸¹, and Y. Unless otherwise specified, each variable within such a formula is defined independently of any other variable, and any variable that occurs more than one time in a formula is defined independently at each occurrence. Thus, for example, if a group is shown to be substituted with 0-2 R*, the group may be unsubstituted or substituted with up to two R* groups, and R* at each occurrence is selected independently from the definition of R*. Also, combinations of substituents and/or variables are permissible only if such combinations result in stable compounds, i.e., compounds that can be isolated, characterized and tested for biological activity.

A “pharmaceutically acceptable salt” of a compound disclosed herein is an acid or base salt that is generally considered in the art to be suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids.

Suitable pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzenesulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is any integer from 0 to 6, i.e. 0, 1, 2, 3, 4, 5 or 6, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two. Generally, the use of nonaqueous media, such as ether, ethyl acetate, ethanol, isopropanol or acetonitrile, is preferred.

It will be apparent that each compound of formula (I) may, but need not, be present as a hydrate, solvate or non-covalent complex. In addition, the various crystal forms and polymorphs are within the scope of the present invention as are prodrugs of the compounds of formula (I) provided herein.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

A “substituent,” as used herein, refers to a molecular moiety that is covalently bonded to an atom within a molecule of interest. For example, a “ring substituent” may be a moiety such as a halogen, alkyl group, haloalkyl group or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e., a compound that can be isolated, characterized and tested for biological activity. When a substituent is oxo, i.e., =O, then 2 hydrogens on the atom are replaced. An oxo group that is a substituent of an aromatic carbon atom results in a conversion of —CH—to —C(═O)— and a loss of aromaticity. For example a pyridyl group substituted by oxo is a pyridone.

The expression “optionally substituted” refers to a group in which one, two, three or more hydrogen atoms may have been replaced independently of each other by the respective substituents.

As used herein, the term “amino acid” refers to any organic acid containing one or more amino substituents, e.g. α-, β- or γ-amino, derivatives of aliphatic carboxylic acids. In the polypeptide notation used herein, e.g. Xaa₅, i.e. Xaa₁Xaa₂Xaa₃Xaa₄Xaa₅, wherein Xaa₁ to Xaa₅ are each and independently selected from amino acids as defined, the left hand direction is the amino terminal direction and the right hand direction is the carboxy terminal direction, in accordance with standard usage and convention.

The term “conventional amino acid” refers to the twenty naturally occurring amino acids, and encompasses all stereomeric isoforms, i.e. D,L-, D- and L-amino acids thereof. These conventional amino acids can herein also be referred to by their conventional three- letter or one-letter abbreviations and their abbreviations follow conventional usage (see, for example, Immunology—A Synthesis, 2^{nd} Edition, E. S. Golub and D. R. Gren, Eds., Sinauer Associates, Sunderland Mass. (1991)).

The term “non-conventional amino acid” refers to unnatural amino acids or chemical amino acid analogues, e.g. α,α-disubstituted amino acids, N-alkyl amino acids, homo-amino acids, dehydroamino acids, aromatic amino acids (other than phenylalanine, tyrosine and tryptophan), and ortho-, meta- or para-aminobenzoic acid. Non-conventional amino acids also include compounds which have an amine and carboxyl functional group separated in a 1,3 or larger substitution pattern, such as P-alanine, y-amino butyric acid, Freidinger lactam, the bicyclic dipeptide (BTD), amino-methyl benzoic acid and others well known in the art. Statine-like isosteres, hydroxyethylene isosteres, reduced amide bond isosteres, thioamide isosteres, urea isosteres, carbamate isosteres, thioether isosteres, vinyl isosteres and other amide bond isosteres known to the art may also be used. The use of analogues or non-conventional amino acids may improve the stability and biological half-life of the added peptide since they are more resistant to breakdown under physiological conditions. The person skilled in the art will be aware of similar types of substitution which may be made. A non limiting list of non-conventional amino acids which may be used as suitable building blocks for a peptide and their standard abbreviations (in brackets) is as follows: α-aminobutyric acid (Abu), L-N-methylalanine (Nmala), α-amino-α-methylbutyrate (Mgabu), L-N-methylarginine (Nmarg), aminocyclopropane (Cpro), L-N-methylasparagine (Nmasn), carboxylate L-N-methylaspartic acid (Nmasp), aniinoisobutyric acid (Aib), L-N-methylcysteine (Nmcys), aminonorbomyl (Norb), L-N-methylglutamine (Nmgln), carboxylate L-N-methylglutamic acid (Nmglu), cyclohexylalanine (Chexa), L-N-methylhistidine (Nmhis), cyclopentylalanine (Cpen), L-N-methylisolleucine (Nmile), L-N-methylleucine (Nmleu), L-N-methyllysine (Nmlys), L-N-methylmethionine (Nmmet), L-N-methylnorleucine (Nmnle), L-N-methylnorvaline (Nmnva), L-N-methylornithine (Nmorn), L-N-methylphenylalanine (Nmphe), L-N-methylproline (Nmpro), L-N-methylserine (Nmser), L-N-methylthreonine (Nmthr), L-N-methyltryptophan (Nmtrp), D-ornithine (Dom), L-N-methyltyrosine (Nmtyr), L-N-methylvaline (Nmval), L-N-methylethylglycine (Nmetg), L-N-methyl-t-butylglycine (Nmtbug), L-norleucine (NIe), L-norvaline (Nva), α-methyl-aminoisobutyrate (Maib), α-methyl-γ-aminobutyrate (Mgabu), D-α-methylalanine (Dmala), α-methylcyclohexylalanine (Mchexa), D-α-methylarginine (Dmarg), α-methylcylcopentylalanine (Mcpen), D-α-methylasparagine (Dmasn), α-methyl-α-napthylalanine (Manap), D-a-methylaspartate (Dmasp), α-methylpenicillamine (Mpen), D-α-methylcysteine (Dmcys), N-(4-aminobutyl)glycine (NgIu), D-α-methylglutamine (Dmgln), N-(2-aminoethyl)glycine (Naeg), D-α-methylhistidine (Dmhis), N-(3-aminopropyl)glycine (Nom), D-α-methylisoleucine (Dmile), N-amino-α-methylbutyrate (Nmaabu), D-α-methylleucine (Dmleu), α-napthylalanine (Anap), D-α-methyllysine (Dmlys), N-benzylglycine (Nphe), D-α-methylmethionine (Dmmet), N-(2-carbamylethyl)glycine (NgIn), D-α-methylornithine (Dmorn), N-(carbamylmethyl)glycine (Nasn), D-α-methylphenylalanine (Dmphe), N-(2-carboxyethyl)glycine (NgIu), D-α-methylproline (Dmpro), N-(carboxymethyl)glycine (Nasp), D-α-methylserine (Dmser), N-cyclobutylglycine (Ncbut), D-α-methylthreonine (Dmthr), N-cycloheptylglycine (Nchep), D-α-methyltryptophan (Dmtrp), N-cyclohexylglycine (Nchex), D-α-methyltyrosine (Dmty), N-cyclodecylglycine (Ncdec), D-α-methylvaline (Dmval), N-cylcododecylglycine (Ncdod), D-N-methylalanine (Dnmala), N-cyclooctylglycine (Ncoct), D-N-methylarginine (Dnmarg), N-cyclopropylglycine (Ncpro), D-N-methylasparagine (Dnmasn), N-cycloundecylglycine (Ncund), D-N-methylaspartate (Dnmasp), N-(2,2-diphenylethyl)glycine (Nbhm), D-N-methylcysteine (Dnmcys), N-(3,3-diphenylpropyl)glycine (Nbhe), D-N-methylglutamine (Dnmgln), N-(3-guanidinopropyl)glycine (Narg), D-N-methylglutamate (Dnmglu), N-(1-hydroxyethyl)glycine (Ntbx), D-N-methylhistidine (Dnmhis), N-(hydroxyethyl))glycine (Nser), D-N-methylisoleucine (Dnmile), N-(imidazolylethyl))glycine (Nhis), D-N-methylleucine (Dnmleu), N-(3-indolylyethyl)glycine (Nhtrp), D-N-methyllysine (Dnnilys), N-methyl-γ-aminobutyrate (Nmgabu), N-methylcyclohexylalanine (Nmchexa), D-N-methylmethionine (Dnmmet), D-N-methylornithine (Dnmorn), N-methylcyclopentylalanine (Nmcpen), N-methylglycine (NaIa), D-N-methylphenylalanine (Dnmphe), N-methylaminoisobutyrate (Nmaib), D-N-methylproline (Dnmpro), N-(1-methylpropyl)glycine (Nile), D-N-methylserine (Dnmser), N-(2-methylpropyl)glycine (Nleu), D-N-methylthreonine (Dnmthr), D-N-methyltryptophan (Dnmtrp), N-(1-methylethyl)glycine (Nval), D-N-methyltyrosine (Dnmtyr), N-methyla-napthylalanine (Nmanap), D-N-methylvaline (Dnmval), N-methylpenicillamine (Nmpen), γ-aminobutyric acid (Gabu), N-(p-hydroxyphenyl)glycine (Nhtyr), L−/−butylglycine (Tbug), N-(thiomethyl)glycine (Ncys), L-ethylglycine (Etg), penicillamine (Pen), L-homophenylalanine (Hphe), L-α-methylalanine (Mala), L-α-methylarginine (Marg), L-α-methylasparagine (Masn), L-α-methylaspartate (Masp), L-α-methyl-t-butylglycine (Mtbug), L-α-methylcysteine (Mcys), L-methylethylglycine (Metg), L-a-methylglutamine (MgIn), L-α-methylglutamate (MgIu), L-α-methylhistidine (Mhis), L-a-methylhomophenylalanine (Mhphe), L-α-methylisoleucine (Mile), N-(2-methylthioethyl)glycine (Nmet), L-α-methylleucine (Mleu), L-α-methyllysine (Mlys), L-α-methylmethionine (Mmet), L-a-methylnorleucine (MnIe), L-α-methylnorvaline (Mnva), L-α-methylornithine (Mom), L-a-methylphenylalanine (Mphe), L-α-methylproline (Mpro), L-α-methylserine (Mser), L-α-methylthreonine (Mthr), L-α-methyltryptophan (Mtrp), L-α-methyltyrosine (Mtyr), L-α-methylvaline (Mval), L-N-methylhomophenylalanine (Nmhphe), N—(N-(2,2-diphenylethyl)carbamylmethyl)glycine (Nnbhm), N—(N-(3,3-diphenylpropyl)carbamylmethyl)glycine (Nnbhe), 1-carboxy-1-(2,2-diphenyl-ethylamino)cyclopropane (Nmbc), L-O-methyl serine (Omser), L-O-methyl homoserine (Omhser).

The expression alkyl refers to a saturated, straight-chain or branched hydrocarbon group that contains from 1 to 20 carbon atoms, preferably from 1 to 10 carbon atoms, e.g. a n-octyl group, especially from 1 to 6, i.e. 1, 2, 3, 4, 5, or 6, carbon atoms, for example a methyl, ethyl, propyl, isopropyl, n-butyl, iso-butyl, sec-butyl, tert-butyl, n-pentyl, iso-pentyl, n-hexyl, or 2,2-dimethylbutyl.

The expression alkenyl refers to an at least partially unsaturated, straight-chain or branched, hydrocarbon group that contains from 2 to 21 carbon atoms, preferably from 2 to 6 carbon atoms, i.e. 2, 3, 4, 5 or 6 carbon atoms, for example an ethenyl (vinyl), propenyl (allyl), iso-propenyl, butenyl, isoprenyl or hex-2-enyl group, or from 11 to 21 carbon atoms, i.e. 11, 12, 13, 14, 15, 16, 17, 18, 19, 20 or 21 carbon atoms, for example a hydrocarbon group comprising a methylene chain interrupted by one double bond as, for example, found in monounsaturated fatty acids or a hydrocarbon group comprising methylene-interrupted polyenes, e.g. hydrocarbon groups comprising two or more of the following structural unit —[CH═CH—CH₂]—, as, for example, found in polyunsaturated fatty acids. Alkenyl groups have one or more, preferably 1, 2, 3, 4, 5, or 6 double bond(s).

The expression alkynyl refer to at least partially unsaturated, straight-chain or branched hydrocarbon groups that contain from 2 to 20 carbon atoms, preferably from 2 to 10 carbon atoms, especially from 2 to 6, i.e. 2, 3, 4, 5 or 6, carbon atoms, for example an ethinyl, propinyl, butinyl, acetylenyl, or propargyl group. Preferably, alkynyl groups have one or two (especially preferably one) triple bond(s).

Furthermore, the terms alkyl, alkenyl and alkynyl refer to groups in which one or more hydrogen atom(s) have been replaced, e.g. by a halogen atom, preferably F or Cl, such as, for example, a 2,2,2-trichloroethyl or a trifluoromethyl group.

The expression heteroalkyl refers to an alkyl, alkenyl or alkynyl group in which one or more, preferably 1, 2 or 3, carbon atoms, have been replaced independently of each other by an oxygen, nitrogen, phosphorus, boron, selenium, silicon or sulfur atom, preferably by an oxygen, sulfur or nitrogen atom. The expression heteroalkyl can also refer to a carboxylic acid or to a group derived from a carboxylic acid, such as, for example, acyl, acylalkyl, alkoxycarbonyl, acyloxy, acyloxyalkyl, carboxyalkylamide or alkoxycarbonyloxy.

Preferably, a heteroalkyl group contains from 1 to 10 carbon atoms and from 1 to 4 hetero atoms selected from oxygen, nitrogen and sulphur (especially oxygen and nitrogen). Especially preferably, a heteroalkyl group contains from 1 to 6, i.e. 1, 2, 3, 4, 5, or 6, carbon atoms and 1, 2 or 3, especially 1 or 2, hetero atoms selected from oxygen, nitrogen and sulphur, especially oxygen and nitrogen.

Examples of heteroalkyl groups are groups of formulae: R^{a}—O—Y^{a}—, R^{a}—S—Y^{a}—, R^{a}—N(R^{b})—Y^{a}—, R^{a}—CO—Y^{a}—, R^{a}—O—CO—Y^{a}—, R^{a}—CO—O—Y^{a}—, R^{a}—CO—N(R^{b})—Y^{a}—, R^{a}—N(R^{b})—CO—Y^{a}—, R^{a}—O—CO—N(R^{b})-Y^{a}-, R^{a}—N(R^{b})—CO—O—Y^{a}—, R^{a}—N(R^{b})—CO—N(R^{a})—Y^{a}—, R^{a}—O—CO—O—Y^{a}—, R^{a}—N(R^{b})—C(═NR^{d})—N(R^{c})—Y^{a}—, R^{a}—CS—Y^{a}—, R^{a}—O—CS—Y^{a}—, R^{a}—CS—O—Y^{a}—, R^{a}—CS—N(R^{b})—Y^{a}—, R^{a}—N(R^{b})—CS—Y^{a}—, R^{a}—O—CS—N(R^{b})-Y^{a}-, R^{a}—N(R^{b})—CS—O—Y^{a}—, R^{a}—N(R^{b})—CS—N(R)—Y^{a}—, R^{a}—O—CS—O—Y^{a}—, R^{a}—S—CO—Y^{a}—, R^{a}—CO—S—Y^{a}—, R^{a}—S—CO—N(R)—Y^{a}—, R^{a}—N(R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}-, wherein R^{a} being a hydrogen atom, a C₁-C₆ alkyl, a C₂-C₆ alkenyl or a C₂-C₆ alkynyl group; R^{b} being a hydrogen atom, a C₁-C₆ alkyl, a C₂-C₆ alkenyl or a C₂-C₆ alkynyl group; R^{c} being a hydrogen atom, a C₁-C₆ alkyl, a C₂-C₆ alkenyl or a C₂-C₆ alkynyl group; R^{d} being a hydrogen atom, a C₁-C₆ alkyl, a C₂-C₆ alkenyl or a C₂-C₆ alkynyl group and Y^{a} being a direct bond, a C₁-C₆ alkylene, a C₂-C₆ alkenylene or a C₂-C₆ alkynylene group, wherein each heteroalkyl group contains at least one carbon atom and one or more hydrogen atoms may be replaced by fluorine or chlorine atoms.

Specific examples of heteroalkyl groups are methoxy, trifluoromethoxy, ethoxy, n-propyloxy, isopropyloxy, butoxy, tert-butyloxy, methoxymethyl, ethoxymethyl, —CH₂CH₂OH, —CH₂OH, methoxyethyl, 1-methoxyethyl, 1-ethoxyethyl, 2-methoxyethyl or 2-ethoxyethyl, methylamino, ethylamino, propylamino, isopropylamino, dimethylamino, diethylamino, isopropylethylamino, methylamino methyl, ethylamino methyl, diisopropylamino ethyl, methylthio, ethylthio, isopropylthio, enol ether, dimethylamino methyl, dimethylamino ethyl, acetyl, propionyl, butyryloxy, acetyloxy, methoxycarbonyl, ethoxycarbonyl, propionyloxy, acetylamino or propionylamino, carboxymethyl, carboxyethyl or carboxypropyl, N-ethyl-N-methylcarbamoyl or N-methylcarbamoyl. Further examples of heteroalkyl groups are nitrile, isonitrile, cyanate, thiocyanate, isocyanate, isothiocyanate and alkylnitrile groups.

The expression alkoxy refers to an alkyl group singular bonded to oxygen.

The expression alkylthio refers to an alkyl group singular bonded to sulfur.

The expressions cycloalkyl and carbocyclic ring refer to a saturated cyclic group of hydrocarbons that contains one or more rings, preferably 1 or 2), and contains from 3 to 14 ring carbon atoms, preferably from 3 to 10, especially 3, 4, 5, 6 or 7 ring carbon atoms, e.g. a cyclopropyl, cyclobutyl, cyclopentyl, spiro[4,5]decanyl, norbornyl, cyclohexyl, decalinyl, bicyclo[4.3.0]nonyl, tetraline, or cyclopentylcyclohexyl group. The expression cycloalkyl refers furthermore to groups in which one or more hydrogen atoms have been replaced by fluorine, chlorine, bromine or iodine atoms or by OH, =O, SH, NH₂, =NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group.

The expression aryl refers to an aromatic group that contains one or more rings containing from 6 to 14 ring carbon atoms, preferably from 6 to 10, especially 6, ring carbon atoms.

The expression heteroaryl refers to an aromatic group that contains one or more rings containing from 5 to 14 ring atoms, preferably from 5 to 10, especially 5 or 6, ring atoms, and contains one or more, preferably 1, 2, 3 or 4, oxygen, nitrogen, phosphorus or sulfur ring atoms, preferably 0, S or N. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl,thiadiazolyl, indolyl, indazolyl, tetrazolyl, pyrazinyl, pyrimidinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (e.g. 3-pyrazolyl) and isoquinolinyl groups. The expression heterocycloalkyl refers to a cycloalkyl group as defined above in which one or more (preferably 1, 2 or 3) ring carbon atoms, each independently, have been replaced by an oxygen, nitrogen, silicon, selenium, phosphorus or sulfur atom (preferably by an oxygen, sulfur or nitrogen atom). A heterocycloalkyl group has preferably 1 or 2 ring(s) containing from 3 to 10 (especially 3, 4, 5, 6 or 7) ring atoms (preferably selected from C, O, N and S). The expression heterocycloalkyl refers furthermore to groups in which one or more hydrogen atoms have been replaced by fluorine, chlorine, bromine or iodine atoms or by OH, =O, SH, ═S, NH₂, =NH, N₃ or NO₂ groups. Examples are a piperidyl, prolinyl, imidazolidinyl, piperazinyl, morpholinyl, urotropinyl, pyrrolidinyl, tetrahydrothiophenyl, tetrahydropyranyl, tetrahydrofuryl or 2-pyrazolinyl group and also lactames, lactones, cyclic imides and cyclic anhydrides.

The expression alkylcycloalkyl refers to a group that contains both cycloalkyl and also alkyl, alkenyl or alkynyl groups in accordance with the above definitions, for example alkylcycloalkyl, cyclo-alkylalkyl, alkylcycloalkenyl, alkenylcycloalkyl and alkynylcycloalkyl groups. An alkylcycloalkyl group preferably contains a cycloalkyl group that contains one or two ring systems having from 3 to 10 (especially 3, 4, 5, 6 or 7) ring carbon atoms, and one or two alkyl, alkenyl or alkynyl groups having 1 or 2 to 6 carbon atoms. The expression aralkyl refers to a group containing both aryl and also alkyl, alkenyl, alkynyl and/or cycloalkyl groups in accordance with the above definitions, such as, for example, an arylalkyl, arylalkenyl, arylalkynyl, arylcycloalkyl, arylcycloalkenyl, alkylarylcycloalkyl and alkylarylcycloalkenyl group. Specific examples of aralkyls are toluene, xylene, mesitylene, styrene, benzyl chloride, o-fluorotoluene, 1H-indene, tetraline, dihydronaphthalene, indanone, phenylcyclopentyl, cumene, cyclohexylphenyl, fluorene and indane. An aralkyl group preferably contains one or two aromatic ring systems (1 or 2 rings) containing from 6 to 10 carbon atoms and one or two alkyl, alkenyl and/or alkynyl groups containing from 1 or 2 to 6 carbon atoms and/or a cyclo-alkyl group containing 5 or 6 ring carbon atoms.

The expression heteroalkylcycloalkyl refers to alkylcycloalkyl groups as defined above in which one or more, preferably 1, 2 or 3, carbon atoms have been replaced independently of each other by an oxygen, nitrogen, silicon, selenium, phosphorus or sulfur atom (preferably by an oxygen, sulfur or nitrogen atom). A heteroalkylcycloalkyl group preferably contains 1 or 2 ring systems having from 3 to 10 (especially 3, 4, 5, 6 or 7) ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being saturated or mono-, di- or tri-unsaturated.

The expression heterocyclic ring refers to heteroaryl group as defined above as well as to a cycloalkyl group or carbocyclic ring as defined above in which one or more (preferably 1, 2 or 3) ring carbon atoms, each independently, have been replaced by an oxygen, nitrogen, silicon, selenium, phosphorus or sulfur atom, preferably by an oxygen, sulfur or nitrogen atom. A heterocyclic ring has preferably 1 or 2 ring(s) containing from 3 to 10, especially 3, 4, 5, 6 or 7 ring atoms, preferably selected from C, O, N and S. Examples are a aziridinyl, oxiranyl, thiiranyl, oxaziridinyl, dioxiranyl, azetidinyl, oxetanyl, thietanyl, diazetidinyl, dioxetanyl, dithietanyl, pyrrolidinyl, tetrahydrofuranyl, thiolanyl, phospholanyl, silolanyl, azolyl, thiazolyl, isothiazolyl, imidazolidinyl, pyrazolidinyl, oxazolidinyl, isoxazolidinyl, thiazolidinyl, isothiazolidinyl, dioxolanyl, dithiolanyl, piperazinyl, morpholinyl, thiopmorpholinyl, trioxanyl, azepanyl, oxepanyl, thiepanyl, homopiperazinyl, or urotropinyl group.

The expression heteroaralkyl refers to an aralkyl group as defined above in which one or more (preferably 1, 2, 3 or 4) carbon atoms, each independently, have been replaced by an oxygen, nitrogen, silicon, selenium, phosphorus, boron or sulfur atom (preferably oxygen, sulfur or nitrogen), that is to say to a group containing both aryl or heteroaryl, respectively, and also alkyl, alkenyl, alkynyl and/or heteroalkyl and/or cycloalkyl and/or heterocycloalkyl groups in accordance with the above definitions. A heteroaralkyl group preferably contains one or two aromatic ring systems (1 or 2 rings) containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms.

Examples are arylheteroalkyl, arylheterocycloalkyl, arylheterocycloalkenyl, arylalkyl-heterocycloalkyl, arylalkenylheterocycloalkyl, arylalkynylheterocycloalkyl, arylalkylheterocyclo-alkenyl, heteroarylalkyl, heteroarylalkenyl, heteroarylalkynyl, heteroarylheteroalkyl, heteroaryl-cycloalkyl, heteroarylcycloalkenyl, heteroarylheterocycloalkyl, heteroarylheterocycloalkenyl, hetero-arylalkylcycloalkyl, heteroarylalkylheterocycloalkenyl, heteroarylheteroalkylcycloalkyl, heteroaryl-heteroalkylcycloalkenyl and heteroarylheteroalkylheterocycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

As already stated above, the expressions cycloalkyl, heterocycloalkyl, alkylcycloalkyl, hetero-alkylcycloalkyl, aryl, heteroaryl, aralkyl and heteroaralkyl also refer to groups in which one or more hydrogen atoms of such groups have been replaced independently of each other by fluorine, chlorine, bromine or iodine atoms or by OH, =O, SH, ═S, NH₂, =NH, N₃ or NO₂ groups.

The general term ring as used herein, unless defined otherwise, includes cycloalkyl groups or carbocyclic rings, heterocyclic rings, aryl groups, and heteroaryl groups.

The expressions “halo”, “halogen”” or “halogen atom” as used herein means fluorine, chlorine, bromine, or iodine, preferably fluorine and/or chlorine.

The expression mono- or disaccharide, and derivatives thereof as used herein means a carbohydrate or sugar belonging to or derived from the group of monosaccharides or disaccharides.

Examples of mono-, disaccharides, and derivatives include glucose, 3-O-methyl-glucose, 1-deoxy-glucose, 6-deoxy-glucose, galactose, mannose, fructose, xylose, ribose, cellobiose, maltose, lactose, gentiobiose, saccharose, trehalose and mannitol, sorbitol and ribitol. Preferably, the saccharides are D-form saccharides, e.g. D-glucose, 3-O-methyl-D-glucose, 1-deoxy-D-glucose, or 6-deoxy-D-glucose, D-galactose, D-mannose.

As used herein a wording defining the limits of a range of length such as, e.g., “from 1 to 5” means any integer from 1 to 5, i.e. 1, 2, 3, 4 and 5. In other words, any range defined by two integers explicitly mentioned is meant to comprise and disclose any integer defining said limits and any integer comprised in said range.

The expression “—C(═O)O-motif” is used herein in order to clearly define a group comprising an sp²-hybridized carbonyl carbon attached (i) to any carbon or hetero atom and (ii) to an oxygen which in turn can be attached to hydrogen or any other chemical atom. The term “carboxyl group” is avoided for the description of a “—C(═O)O-motif” because it could be mistaken as describing the carboxylic acid only.

The term “in alpha position” is used to describe a directly adjacent position, while the term “in beta position” indicates a neighboring position of an atom or group A and an atom or group B, characterized in that one further atom or group is localized between A and B.

As used herein, the term oxamide refers to the arbitrarily substituted organic compound comprising 2 carbonyl carbons and two nitrogens, which compound is an arbitrarily substituted diamide derived from any oxalic acid.

Those skilled in the art will readily recognize that some of the n-3 PUFA analogues of general formula (I) of the present invention represent “bioisosteres” of the naturally occurring epoxymetabolites produced by cytochrome P450 (CYP) enzymes from omega-3 (n-3) polyun-saturated fatty acids (PUFAs). A bioisostere is a compound resulting from the exchange of an atom or of a group of atoms with an alternative, broadly similar, atom or group of atoms, thereby creating a new compound with similar biological properties to the parent compound. Bioisosterism has, for example, been used by medicinal chemists for improving desired biological or physical properties of a compound, e.g. to attenuate toxicity, modify activity, alter pharmacokinetics and/or metabolism of a compound. For example, the replacement of a hydrogen atom with fluorine at a site of metabolic oxidation in a compound may prevent such metabolism from taking place. Because fluorine is similar in size to the hydrogen atom the overall topology of the molecule is not significantly affected, leaving the desired biological activity unaffected. However, with a blocked pathway for metabolism, said compound may have a longer half-life. Another example is the bioisosteric replacement of carboxylic acid groups which has resulted in analogues showing improved bioavailability, enhanced blood-brain barrier penetration, increased activity, better chemical stability and/or selectivity towards the target (see, e.g. the textbook, “The practice of medicinal chemistry”, edited by Camille Georges Wermuth, 3^{rd} edition, Academic Press, 2008, e.g. p. 303-310; Ballatore C. et al. “Carboxylic Acid (Bio)Isosteres in Drug Design”, ChemMedChem 8, 385-395 (2013)). Further, bioisosterism can also be used to provide a “prodrug” of a compound, i.e. a compound that is initially administered to a subject or patient in an inactive (or less active) form, and then becomes modified in vivo to its active form through the normal metabolic processes of the body. For example, conjugation of a compound with lipid and/or sugar units has resulted in analogues (prodrugs) showing increased drug delivery compared to the parent compound (see, e.g. Wong A. and Toth I. “Lipid, Sugar and Liposaccharide Based Delivery Systems”, Current Medicinal Chemistry 8, 1123-1136 (2001)).

The n-3 PUFA analogues of general formula (I) of the present invention can be prepared in a number of ways well known to one skilled in the art of organic synthesis. For example, the compounds of the present invention can be synthesized according to the general reaction schemes shown below using synthetic methods known in the art of synthetic organic chemistry, or variations thereon as appreciated by those skilled in the art. Unless indicated otherwise, all variables, e.g. n, k, R² (also referred to as R₂), R⁶, R⁷, R⁸, R⁴¹, R⁴², R⁴⁴ and R⁴⁵, have the above defined meaning. As starting materials reagents of standard commercial grade can be used without further purification, or can be readily prepared from such materials by routine methods. Those skilled in the art of organic synthesis will recognize that starting materials and reaction conditions may be varied including additional steps employed to produce compounds for use encompassed by the present invention.

The compounds of the present invention are effective for treating, reducing the risk of developing or preventing a disorder associated with an elevated GDF-15 plasma concentration, preferably wherein the GDF-15 plasma concentration is at least 1000 ng/L, preferably wherein the disease associated with an elevated GDF-15 plasma concentration is selected from a cardiovascular disease and a metabolic disease

In one embodiment the cardiovascular disease is selected from atrial fibrillation, bleeding risk associated with atrial fibrillation, a coronary artery disease (CAD), angina, myocardial infarction, stroke, heart failure, hypertensive heart disease, rheumatic heart disease, cardiomyopathy, congenital heart disease, valvular heart disease, carditis, aortic aneurysm, peripheral artery disease, thromboembolic disease and venous thrombosis. In a preferred embodiment the cardiovascular disease is selected from atrial fibrillation, bleeding risk associated with atrial fibrillation, heart failure, a coronary artery disease (CAD) and a peripheral artery disease. In a more preferred embodiment the cardiovascular disease is atrial fibrillation or diseases associated therewith (e.g. bleeding risk). In a more preferred embodiment the cardiovascular disease is atrial fibrillation.

In a preferred embodiment the cardiovascular disease (or any of the above explicitly disclosed cardiovascular diseases) is associated with an elevated GDF-15 plasma concentration, wherein the GDF-15 plasma concentration preferably is at least 500 ng/L, 750 ng/L, 900 ng/L, 1000 ng/L, 1200 ng/L or 1500 ng/L, preferably at least 900 ng/L, 1000 ng/L, 1200 ng/L, more preferably at least 1000 ng/L.

In one embodiment the metabolic disease is selected from diabetes mellitus, dyslipidemia and metabolic syndrome.

In a preferred embodiment the metabolic disease (or any of the above disclosed explicit diseases) is associated with an elevated GDF-15 plasma concentration, wherein the GDF-15 plasma concentration preferably is at least 500 ng/L, 750 ng/L, 900 ng/L, 1000 ng/L, 1200 ng/L or 1500 ng/L, preferably at least 900 ng/L, 1000 ng/L, 1200 ng/L, more preferably at least 1000 ng/L.

In one embodiment the disorder associated with elevated GDF-15 plasma concentration is not a cardiovascular disease.

In one embodiment the disorder associated with elevated GDF-15 plasma concentration is not a metabolic disease.

In one embodiment a composition, preferably a pharmaceutical composition, is provided that comprises the compound of the present invention for the same use as exemplified herein for the compound of the present invention.

In a preferred embodiment, the compound or composition for use according to the invention is administered orally, topically, subcutaneously, intravitrealy, intramuscularly, intraperitoneally, intravenously, or intranasally, preferably orally or intraveneously, more preferably orally or intraperitoneally. The preferred delivery route of ocular medications to the eye for the treatment of an ophthalmological disorder is topical, local ocular (e.g., subconjunctival, intravitreal, retrobulbar, intracameral), and systemic. The latter is preferably achieved through oral, intramuscular or intravenous administration.

It is further preferred that the compound or composition for use according to the invention is a dosage form selected from the group consisting of a spray, an aerosol, a foam, an inhalant, a powder, a tablet, a capsule, a soft gelatin capsule, a tea, a syrup, a granule, a chewable tablet, a salve, a cream, a gel, a suppository, a lozenge, a liposome composition and a solution suitable for injection.

The composition for use according to the invention may further comprise at least one compound of formula (I) and, optionally, one or more carrier substances, e.g. cyclodextrins such as hydroxypropyl p-cyclodextrin, micelles or liposomes, excipients and/or adjuvants. It may additionally comprise, for example, one or more of water, buffers such as, e.g., neutral buffered saline or phosphate buffered saline, ethanol, mineral oil, vegetable oil, dimethylsulfoxide, carbohydrates such as e.g., glucose, mannose, sucrose or dextrans, mannitol, proteins, adjuvants, polypeptides or amino acids such as glycine, antioxidants, chelating agents such as EDTA or glutathione and/or preservatives. Furthermore, one or more other active ingredients may, but need not, be included compositions for us provided herein. For instance, the compounds of the invention may advantageously be employed in combination with direct thrombin inhibitors, statins, RAS drugs, beta blocking agents, diuretics, direct factor Xa inhibitors, vitamin K antagonists, calcium channel blockers and metformin.

The compositions for use may be formulated for any appropriate route of administration, including, for example, topical such as, e.g., transdermal or ocular, oral, buccal, nasal, vaginal, rectal or parenteral administration. The term parenteral as used herein includes subcutaneous, intradermal, intravascular such as, e.g., intravenous, intramuscular, spinal, intracranial, intrathecal, intraocular, periocular, intraorbital, intrasynovial, intraperitoneal and local ocular (e.g., subconjunctival, intravitreal, retrobulbar, intracameral) injection, as well as any similar injection or infusion technique. In certain embodiments, compositions in a form suitable for oral use are preferred. Such forms include, for example, tablets, troches, lozenges, aqueous or oily suspensions, dispersible powders or granules, emulsion, hard or soft capsules, or syrups or elixirs. Within yet other embodiments, compositions provided herein may be formulated as a lyophilizate.

Compositions intended for oral use may further comprise one or more components such as sweetening agents, flavoring agents, coloring agents and/or preserving agents in order to provide appealing and palatable preparations. Tablets contain the active ingredient in admixture with physiologically acceptable excipients that are suitable for the manufacture of tablets. Such excipients include, for example, inert diluents such as, e.g., calcium carbonate, sodium carbonate, lactose, calcium phosphate or sodium phosphate, granulating and disintegrating agents such as, e.g., corn starch or alginic acid, binding agents such as, e.g., starch, gelatin or acacia, and lubricating agents such as, e.g., magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed. Methods for preparing such compositions are known (see, for example, H. C. Ansel and N. G. Popovish, Pharmaceutical Dosage Forms and Drug Delivery Systems, 5th ed., Lea and Febiger (1990)).

Formulations for oral use may also be presented as hard gelatin capsules wherein the active ingredient is mixed with an inert solid diluent such as, e.g., calcium carbonate, calcium phosphate or kaolin, or as soft gelatin capsules wherein the active ingredient is mixed with water or an oil medium such as, e.g., peanut oil, liquid paraffin or olive oil.

Aqueous suspensions contain the active ingredient(s) in admixture with excipients suitable for the manufacture of aqueous suspensions. Such excipients include suspending agents such as, e.g., sodium carboxymethylcellulose, methylcellulose, hydropropylmethylcellulose, sodium alginate, polyvinylpyrrolidone, gum tragacanth and gum acacia; and dispersing or wetting agents such as, e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate. Aqueous suspensions may also comprise one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil such as, e.g., arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavoring agents may be added to provide palatable oral preparations. Such suspensions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavoring and coloring agents, may also be present.

Compositions for use may also be in the form of oil-in-water emulsions. The oily phase may be a vegetable oil such as, e.g., olive oil or arachis oil, a mineral oil such as, e.g., liquid paraffin, or a mixture thereof. Suitable emulsifying agents include naturally-occurring gums such as, e.g., gum acacia or gum tragacanth, naturally-occurring phosphatides such as, e.g., soy bean lecithin, and esters or partial esters derived from fatty acids and hexitol, anhydrides such as, e.g., sorbitan monoleate, and condensation products of partial esters derived from fatty acids and hexitol with ethylene oxide such as, e.g., polyoxyethylene sorbitan monoleate. An emulsion may also comprise one or more sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

Compounds for use according to the invention may be formulated for local or topical administration, such as for topical application to the skin or mucous membranes, such as in the eye. Formulations for topical administration typically comprise a topical vehicle combined with active agent(s), with or without additional optional components. Suitable topical vehicles and additional components are well known in the art, and it will be apparent that the choice of a vehicle will depend on the particular physical form and mode of delivery. Topical vehicles include water; organic solvents such as alcohols such as, e.g., ethanol or isopropyl alcohol or glycerin; glycols such as, e.g., butylene, isoprene or propylene glycol; aliphatic alcohols such as, e.g., lanolin; mixtures of water and organic solvents and mixtures of organic solvents such as alcohol and glycerin; lipid-based materials such as fatty acids, acylglycerols including oils, such as, e.g., mineral oil, and fats of natural or synthetic origin, phosphoglycerides, sphingolipids and waxes; protein-based materials such as collagen and gelatin; silicone-based materials, both non-volatile and volatile; and hydrocarbon-based materials such as microsponges and polymer matrices. A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale-The Extra Pharmacopoeia (Pharmaceutical Press, London 1993) and Martin (ed.), Remington's Pharmaceutical Sciences. Formulations may comprise microcapsules, such as hydroxymethylcellulose or gelatin-microcapsules, liposomes, albumin microspheres, microemulsions, nanoparticles or nanocapsules.

A topical formulation for use may be prepared in a variety of physical forms including, for example, solids, pastes, creams, foams, lotions, gels, powders, aqueous liquids, emulsions, sprays, eye-drops and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form; solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels and often do not contain emulsifiers. Liquid topical products often contain solvents, emulsifiers, moisturizers, emollients, fragrances, dyes/colorants, preservatives and other active ingredients that increase or enhance the efficacy of the final product.

Suitable emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or non-ionic gums such as hydroxyethylcellulose, xanthan gum, magnesium aluminum silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Suitable preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerin, propylene glycol, and butylene glycol. Suitable emollients include lanolin alcohol, lanolin, lanolin derivatives, cholesterol, petrolatum, isostearyl neopentanoate and mineral oils. Suitable fragrances and colors include, but are not limited to, FD&C Red No. 40 and FD&C Yellow No. 5. Other suitable additional ingredients that may be included in a topical formulation include, but are not limited to, abrasives, absorbents, anti-caking agents, anti-foaming agents, anti-static agents, astringents such as, e.g., witch hazel, alcohol and herbal extracts such as chamomile extract, binders/excipients, buffering agents, chelating agents, film forming agents, conditioning agents, propellants, opacifying agents, pH adjusters and protectants.

An example of a suitable topical vehicle for formulation of a gel is: hydroxypropylcellulose (2.1%); 70/30 isopropyl alcohol/water (90.9%); propylene glycol (5.1%); and Polysorbate 80 (1.9%). An example of a suitable topical vehicle for formulation as a foam is: cetyl alcohol (1.1%); stearyl alcohol (0.5%); Quaternium 52 (1.0%); propylene glycol (2.0%); Ethanol 95 PGF3 (61.05%); deionized water (30.05%); P75 hydrocarbon propellant (4.30%). All percents are by weight.

Typical modes of delivery for topical compositions include application using the fingers; application using a physical applicator such as a cloth, tissue, swab, stick or brush; spraying including mist, aerosol or foam spraying; dropper application; sprinkling; soaking; and rinsing. Controlled release vehicles can also be used, and compositions may be formulated for transdermal administration as a transdermal patch.

A composition for use may be formulated as inhaled formulations, including sprays, mists, or aerosols. Such formulations are particularly useful for the treatment of asthma or other respiratory conditions. For inhalation formulations, the compounds provided herein may be delivered via any inhalation methods known to those skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent, e.g., isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient.

Formulations or compositions suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable powder compositions include, by way of illustration, powdered preparations of the active ingredient thoroughly intermixed with lactose or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation.

Compositions for use may also be prepared in the form of suppositories such as e.g., for rectal administration. Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Compositions for use may be formulated as sustained release formulations such as, i.e., a formulation such as a capsule that creates a slow release of modulator following administration. Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulator release. The amount of modulator contained within a sustained release formulation depends upon, for example, the site of implantation, the rate and expected duration of release and the nature of the condition to be treated or prevented.

It will be understood, however, that the specific dose level for any particular patient will depend upon a variety of factors including the activity of the specific compound employed, the age, body weight, general health, sex, diet, time of administration, route of administration, and rate of excretion, drug combination, i.e. other drugs being used to treat the patient, and the severity of the particular disease undergoing therapy.

Preferred compounds of the invention will have certain pharmacological properties. Such properties include, but are not limited to oral bioavailability, such that the preferred oral dosage forms discussed above can provide therapeutically effective levels of the compound in vivo.

n-3 PUFA derivatives provided herein are preferably administered to a patient such as, e.g., a human, orally or parenterally, and are present within at least one body fluid or tissue of the patient.

As used herein, the term “treatment” encompasses both any type of disease-modifying treatment and including symptomatic treatment, i.e., a treatment after the onset of symptoms, either of which may be prophylactic. However, disease-modifying treatment may involve administration before the onset of symptoms, in order to prevent, at least delay or reduce the severity of symptoms after onset. A disease-modifying treatment may also be therapeutic, i.e., after the onset of symptoms, in order to reduce the severity and/or duration of symptoms. A treatment after onset of symptoms may also simply involve stopping progressing of the disease (stable disease). In certain embodiment, the n-3 PUFA derivatives provided herein are administered prophylactically, i.e., before the onset of the disease and/or symptoms, ideally, but not necessarily, to actually prevent the diseases and/or symptoms. It is to be understood that the term prophylaxis and prophylactic in the context of the present invention, simply describes that the compound(s) of the present invention are administered before the onset of symptoms. A prophylactic administration may an administration before the onset of symptoms that are clearly associated with a disease discussed herein: the n-3 PUFA derivatives provided herein may, e.g., be administered to a subject prophylactically when he or she displays certain conditions that may indicate a propensity to develop one of the conditions or diseases that can be treated with one of the n-3 PUFA derivatives of the present invention. Such indicative conditions are, e.g. high blood pressure or diabetes. Such a prophylactic treatment is called primary prophylaxis. In another embodiment, the n-3 PUFA derivatives provided herein may be administered to a subject prophylactically when he or she has previously suffered from a condition or disease that can be treated with the n-3 PUFA derivatives of the present invention, but currently does not display any symptoms. Such a prophylactic treatment is called secondary prophylaxis. Patients receiving the n-3 PUFA derivatives for the purpose of primary or secondary prophylaxis are considered to be in need of such a treatment. Patients may include but are not limited to mammals, especially humans, domesticated companion animals such as dogs, cats, horses, and livestock such as cattle, pigs, sheep, with dosages as described herein.

The activity of the n-3 PUFA analogues according to the invention can, for example, be determined in appropriate in vitro and/or in vivo assays. For instance, the biological activity of the n-3 PUFA analogues according to the present invention may be determined using the established cell model of Kang and Leaf (Proc Natl Acad Sci USA, 1994. 91 (21): p. 9886-90.) known to those skilled in the art.

The following figure and examples serve to illustrate the invention and are not intended to limit the scope of the invention as described in the appended claims.

### FIGURES

FIG. 1: shows the layout of the clinical study described in example 2

FIG. 2: FIG. 2 shows GDF-15 plasma concentration at baseline (V3) in relation to age of (A) all patients of the clinical study of example 2 as well as the subgroups of patients with GDF-15 plasma concentration at baseline (V3) (B) below 1000 ng/L or (C) above 1000 ng/L.

FIG. 3: FIG. 3 shows that GDF-15 plasma concentrations in the patient subgroup with baseline concentrations above 1000 ng/L (right panel) were significantly reduced by treatment with Compound-02.

FIG. 4: FIG. 4 shows changes in GDF-15 plasma concentration from baseline to end-of-treatment (3 months). Compound-02 reduced GDF-15 plasma concentration significantly and dose-dependently by 30% vs. placebo

FIG. 5: FIG. 5 shows a significant reduction in clinically relevant biomarkers hs-CRP, PTX-3, IL-6, MMP-1, MMP-9 and NT-proBNP across all dosing groups.

FIG. 6: FIG. 6 shows within the GDF-15 ≥1000 ng/L group of patients that Compound-02 dose-dependently reduced AF recurrence.

FIG. 7: FIG. 7 shows that in LDLr−/− mice kept on a high fat diet (HFD) (12 weeks) Compound-02 prevented an increase of circulating levels of PTX-3 as a marker of plaque-instability. Furthermore, Compound-02 treatment significantly reduced lesion size in whole aorta by 55%.

FIG. 8: FIG. 8 shows changes in IL-6 plasma concentration from baseline to end-of-treatment (3 months). Compound-02 reduced IL-6 plasma concentration significantly and dose-dependently by 30% vs. placebo.

FIG. 9: FIG. 9 shows changes in PTX-3 plasma concentration from baseline to end-of-treatment (3 months). Compound-02 reduced PTX-3 plasma concentration significantly and dose-dependently by 55% vs. placebo.

FIG. 10: FIG. 10 shows changes in hsCRP plasma concentration from baseline to end-of-treatment (3 months). Compound-02 reduced hsCRP plasma concentration significantly.

### Example 1 Synthesis of Compounds

The synthesis of the compounds of the invention is illustrated in patent applications WO2010/081683 A1, WO2015/110262 A1, WO2017/013264 A1 and WO2017/168007 A1.

The exemplary synthesis of compounds of the invention is disclosed below. The skilled person would know how to synthesize the other compounds of the present invention by following the below route of synthesis and the further synthesis instructions disclosed in above patent applications, i.e. WO2010/081683 A1, WO2015/110262 A1, WO2017/013264 A1 and WO2017/168007 A1

### Compound 1 (Comp-01)

Synthesis of compound 1 (Comp-01) was analogous to synthesis of compound 3 (Comp-03), while the urea-group was introduced following the synthetic route described in patent application WO2010/081683 (example 13).

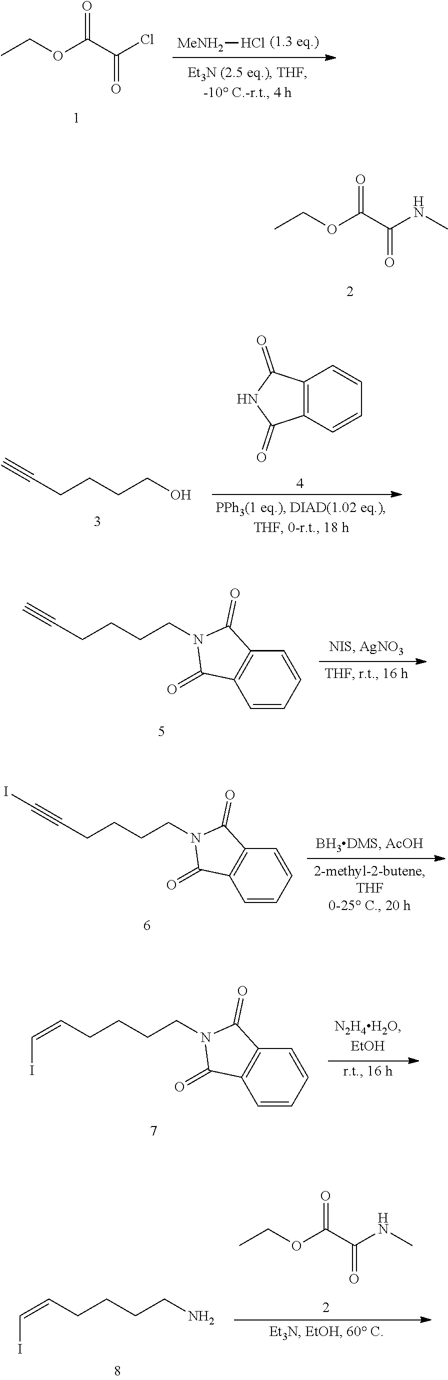

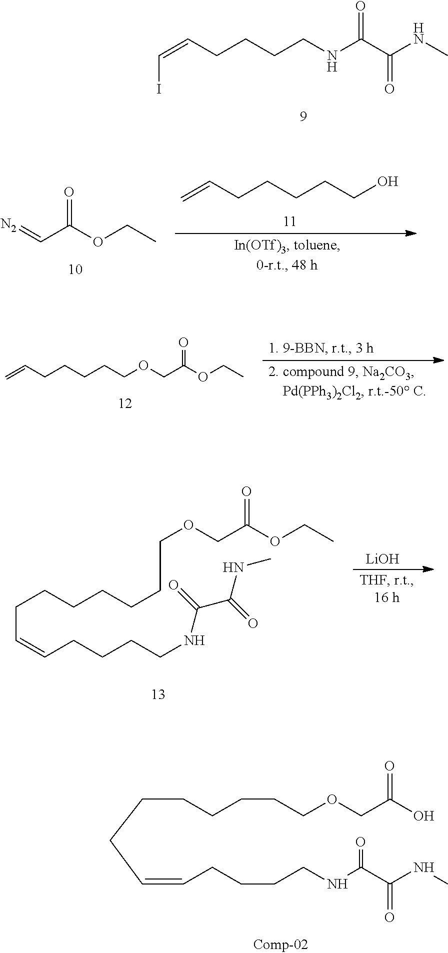

### General Method

NMR spectra were recorded on Bruker Avance 400 MHz for ¹HNMR and 100 MHz for ¹³CNMR. LCMS were taken on a quadrupole Mass Spectrometer on Shimadzu LCMS 2010 (Column: sepax ODS 50×2.0 mm, 5 um) or Agilent 1200 HPLC, 1956 MSD (Column: Shim-pack XR-ODS 30×3.0 mm, 2.2 um) operating in ES (+) ionization mode. Chromatographic purifications were by flash chromatography using 100˜200 mesh silica gel. Anhydrous solvents were pre-treated with 3A MS column before used. All commercially available reagents were used as received unless otherwise stated.

### General Procedure for Preparation of Compound 2

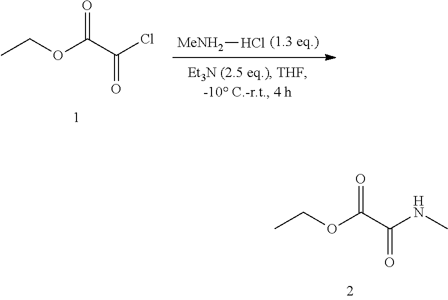

| | | | | | | Other |
|---|---|---|---|---|---|---|
| Reagent | MW. | Amount | | Mmol | ratio | Info. |
| Compound 1 | 136.53 | 100 | g | 732.44 | 1 | |
| MeNH₂—HCl | 67.52 | 64.29 | g | 952.17 | 1.3 | |
| Et₃N | 101.19 | 185.29 | g | 1830 | 2.5 | |
| THF | | 2 | L | | | |
| Product | 131.13 | 70 | g | 533.82 | | Yield: |

| | |
|---|---|
| (compound 2) | 73% |

Methanamine (64.29 g, 952.17 mmol, 1.30 Eq) in 500 mL THF was added Et₃N (75 g, 732.44 mmol), the solution was added to Compound 1 (100.00 g, 732.44 mmol, 1.00 eq), Et₃N (111 g, 1.1 mol) in THF (1.5 L) at −10° C. And the mixture was stirred at 25° C. for 16 h. Then the mixture was filtered, the filtrate was washed with 2N HCl (500 mL), extracted with EA (300 mL*4), concentrated and purified by silica gel (PE:EA=3:1 to 1:1) to afford Compound 2 (70.00 g, 533.82 mmol, 72.88% yield) as a yellow oil.

TLC Information (PE:EtOAc=2:1); R_{f}(Comp-02)=0.39; LCMS: ET2662-1-P1A (M+H⁺): 131.7; ¹H NMR (CDCl₃, 400 MHz) 4.36˜4.24 (q, J=8 Hz, 2H), 2.93˜2.85 (d, J=4 Hz, 3H), 1.38˜1.30 (t, J=8 Hz, 3H).

### Example 2: Efficacy of Compound-02 in a Clinical Phase II Study in Patients with Persistent Atrial Fibrillation (AF)

In a clinical phase II study 119 patients with persistent atrial fibrillation were investigated with regard to the effect of treatment with Compound-02 (structure of general formula (VI)). The patients were divided into a placebo group and three treatment groups with different dosing of Compound-02. The low, medium and high dosing groups received an oral once daily dosing of 4 mg, 12 mg or 24 mg of Compound-02, respectively.

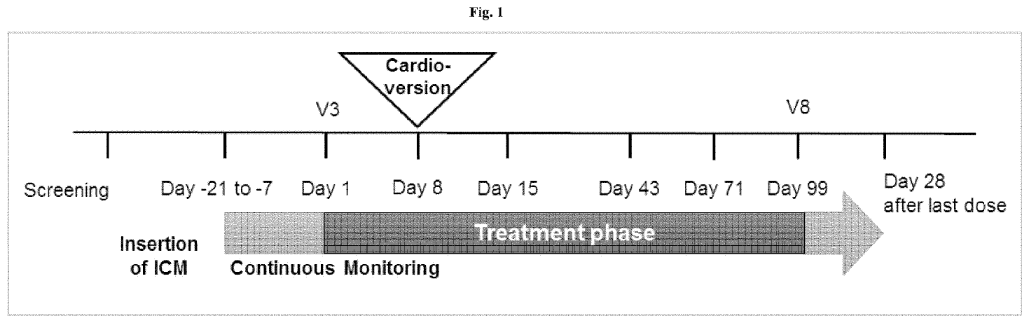

The study participants received an implantable cardiac monitor (ICM) at least one week prior to start of treatment with Compound-02. The treatment with Compound-02 was maintained for a total period of three months (i.e. Day 1 to Day 99) and started at least 1 week prior to direct current cardioversion (DCC) (see FIG. 1 for details of study protocol).

The study revealed a good safety profile of Compound-02 and no changes in the ECG or other types of arrhythmias were observed in study participants. All study groups had a high treatment compliance.

Plasma samples were taken from the patients at different study visits. Biomarkers were analyzed at baseline (V3) and at the end of treatment (V8). For 119 study participants complete sets of biomarkers could be obtained. The analysis revealed a consistent reduction of biomarkers between baseline (V3) and end of treatment (V8), in particular GDF-15, IL-6 and PTX-3 were significantly reduced.

GDF-15 concentrations were measured in EDTA treated plasma samples with a solid phase sandwich ELISA (Human GDF-15 Quantikine ELISA kit, R&D Systems) using an immobilized mouse monoclonal anti-GDF-15 and a horseradish peroxidase-conjugated monoclonal anti-GDF-15 antibody. The substrates for the enzymatic reaction are hydrogen peroxide and tetramethylbenzidine. The reaction is stopped by the addition of sulfuric acid. The reaction product is measured spectrophotometrically at 450 nm (Sunrise Absorbance Reader, TECAN).

Subgroup of Patients with Pathophysiological GDF-15 Plasma Concentration

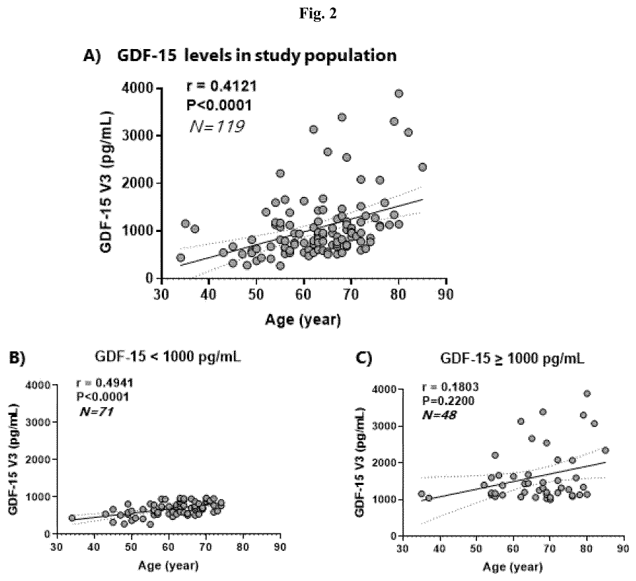

GDF-15 levels in the total study population showed an age dependent increase over the whole group of study participants (correlation r=0.4121, p<0.0001; see FIG. 2A). However, a subgroup of patients showed pathophysiologically high GDF-15 plasma concentrations of ≥1000 ng/L. Within this subgroup of patients no significant correlation between GDF-15 plasma concentration and age existed (see FIG. 2C), whereas the subgroup of patients with GDF-15 plasma concentration of <1000 ng/L demonstrated a significant corellation with age (see FIG. 2B). This indicates that the pathophysiologically high GDF-15 plasma concentrations are caused by the disease and are not merely a result of age as in the group of patients with lower GDF-15 plasma concentration.

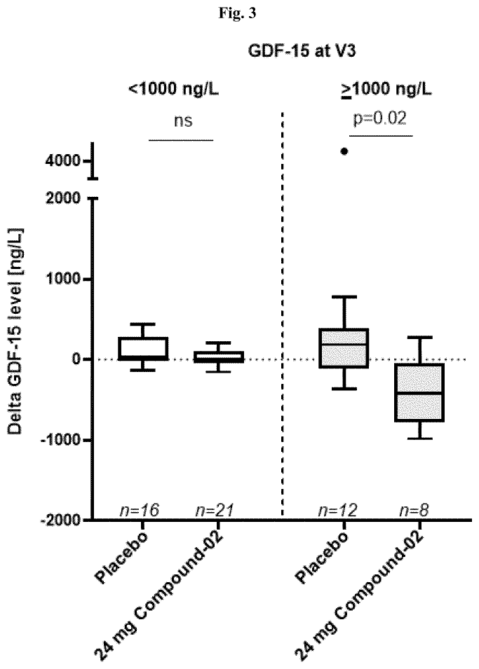

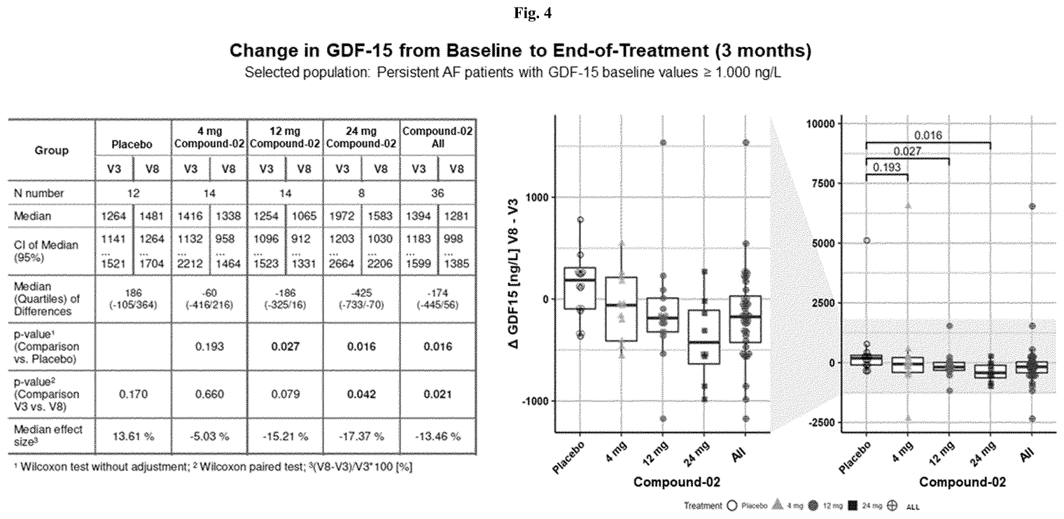

In the patient subgroup with high GDF-15 levels (i.e. GDF-15 plasma concentration ≥1000 ng/L) at baseline visit (V3) a significant decrease in GDF-15 levels could be observed at the end of treatment (V8) with Compound-02 compared to the placebo treated group (p=0.02; see FIG. 3 right panel). In contrast the patient subgroup with a GDF-15 plasma concentration of <1000 ng/L at the baseline visit did not show a significant change in GDF-15 plasma concentration at the end of treatment (see FIG. 3 left panel). The observed decrease of GDF-15 plasma concentration was also dose-dependent as can be appreciated from FIG. 4.

This suggests an effect of Compound-02 in this particular subgroup of patients, i.e. those with a pathophysiological GDF-15 plasma concentration of ≥1000 ng/L.

The subgroup with pathophysiologically increased GDF-15 plasma concentration had also significant difference in same clinical characteristics compared to the group of patients with lower GDF-15 plasma concentration as disclosed in Table 1 below. The subgroup with GDF-15 plasma concentration of ≥1000 ng/L was further associated with a higher age and heart rate. Furthermore, the higher CHA2DS2-VASc score indicates a higher stroke risk. The lower glomerular filtration rate (GFR) is likely the result of the increased age in this group of patients.

TABLE 1
Clinical baseline-results for study population, data are presented
as mean ± SD, median (quartiles), or frequency (%).

| GROUP | GDF15 <1000 | | GDF15 ≥1000 | | P |
|---|---|---|---|---|---|
| N number | 71 | | 48 | | |
| Age | 63.0 | (56.0/67.0) | 68.0 | (58.2/74.2) | 0.004 |
| Sex | | | | | 0.083 |
| F | 22 | (31.0%) | 23 | (47.9%) | |
| M | 49 | (69.0%) | 25 | (52.1%) | |
| Body Mass Index V2 [kg/m2] | 30.8 ± 4.9 | | 30.6 ± 6.2 | | 0.863 |
| Systolic Blood Pressure V3 [mmHg] | 128 ± 9 | | 126 ± 13 | | 0.514 |
| Diastolic Blood Pressure V3 [mmHg] | 81.0 ± 5.7 | | 79.5 ± 9.6 | | 0.348 |
| Heart Rate V3 [beats/min] | 82.9 ± 13.2 | | 89.3 ± 14.2 | | 0.012 |
| Left Vent. Ejec. Frac. at Screening [%] | 56.0 | (51.2/62.0) | 57.0 | (51.0/61.1) | 0.967 |
| Left Atrium Size at Screening [mm] | 45.0 | (42.0/48.0) | 45.0 | (43.0/48.0) | 0.570 |
| CHA2DS2-VASc score | 3.00 | (2.00/4.00) | 4.00 | (3.00/5.00) | 0.004 |
| Glomerular Filtration Rate V3 | 75.7 | (61.2/86.6) | 65.8 | (52.1/78.9) | 0.031 |

[mL/min/1.73 m2]

Effect of Treatment with Compound-02 on Atrial Fibrillation

The subgroup of patients with a pathophysiological GDF-15 plasma concentration of ≥1000 ng/L was further investigated with regard to other effects caused by treatment with Compound-02. Therefore a panel of biomarkers known to be disease relevant was measured and compared across the period of treatment.

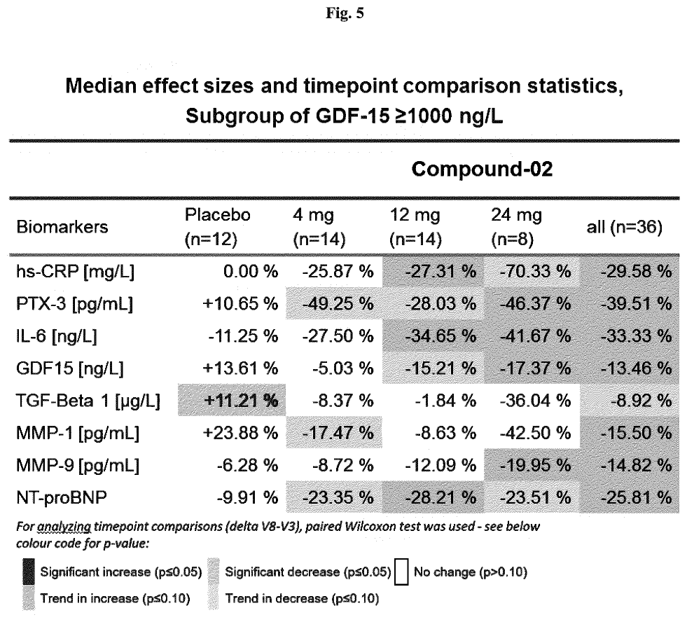

In addition to the decrease of GDF-15 plasma concentration a significant decrease of plasma concentrations at the end of treatment was observed for hs-CRP, PTX-3, IL-6, MMP-1, MMP-9 and NT-proBNP across all dosing groups (see FIG. 5). The reduction of further clinically relevant markers, in particular PTX-3 and IL-6, indicates a therapeutic effect of Compound-02 at least in the subgroup of patients with a pathophysiological GDF-15 plasma concentration of ≥1000 ng/L.

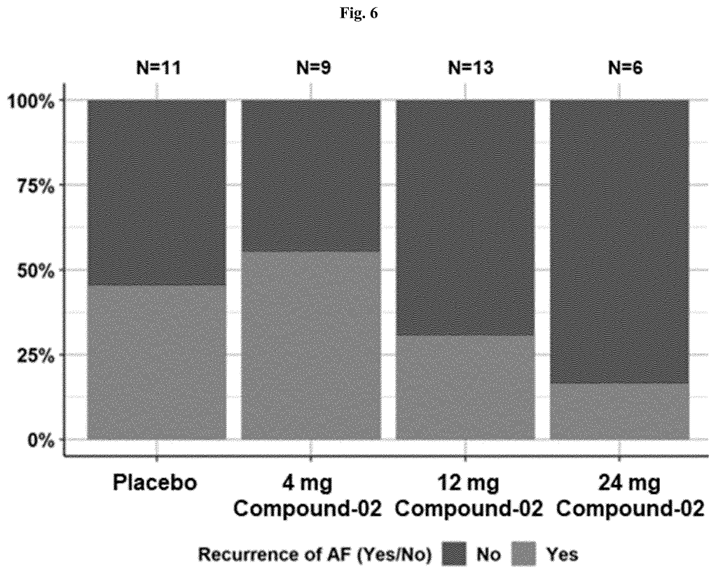

Indeed in the subgroup of patients with a GDF-15 plasma concentration of ≥1000 ng/L a significant reduction of atrial fibrillation (AF) recurrence was observed (see FIG. 6). Furthermore, the reduction of AF recurrence after electrical cardioversion was dose-dependent. This demonstrates a therapeutic effect of Compound-02 in patients with persistent AF, in particular in patients with a GDF-15 plasma concentration of ≥1000 ng/L prior to start of treatment.

### Example 3: Anti-Atherosclerotic Activity of Compound-02

Effect of Treatment with Compound-02 on Coronary Artery Disease

The inventors further investigated a possible therapeutic effects of Compound-02 in the treatment of coronary artery disease (CAD) by lowering GDF-15 levels. Pentraxin-3 (PTX-3) is a known biomarker of cardiac inflammation produced by macrophages and vascular smooth muscle cells, particularyl in the region of the atherosclerotic plaque. PTX-3 is further regarded as a marker of plaque-instability (Soeki et al., J Cardiol. 58:151-157. 2011) and is therefore of clinical relevance in the context of coronary artery disease.

Furthermore, the study of Example 2 showed that Compound-02 mediated lowering of GDF-15 is correlated with lowering of important biomarkers of systemic chronic inflammation (hs-CRP) and artherosclerotic plaque vulnerability (PTX-3) as well as improved lipid metabolism (HDL/LDL ratio) (see Table 2 below). Further, GDF-15 lowering correlated with reduction of NT-proBNP (0.368), a risk stratification marker for patients with acute coronary syndrome.

TABLE 2
Timepoint comparison baseline (V3) vs. end of treatment
(V8); GDF15 ≥1.000 ng/L: Compound-02, all dosed groups
together; Spearman Rank Correlations delta V8-V3 (n = 15)

| | hs-CRP | IL-6 | GDF15 | PTX-3 | HDL/LDL |
|---|---|---|---|---|---|
| | V8-V3 | V8-V3 | V8-V3 | V8-V3 | V8-V3 |
| Var | [mg/L] | [ng/L] | [ng/L] | [pg/mL] | [mmol/L] |
| hs-CRP | 1 | | | | |
| V8-V3 [mg/L] | | | | | |
| IL-6 | 0.719 | 1 | | | |
| V8-V3 [ng/L] | | | | | |
| GDF15 | 0.531 | 0.313 | 1 | | |
| V8-V3 [ng/L] | | | | | |
| PTX-3 | 0.388 | 0.293 | 0.482 | 1 | |
| V8-V3 [pg/mL] | | | | | |
| HDL/LDL | −0.434 | −0.288 | −0.357 | 0.161 | 1 |
| V8-V3 [mmol/L] | | | | | |

Effect of Treatment with Compound-02 in Animal Model of Atherosclerosis

The effect of Compound-02 was investigated in mice with a disrupted low-density lipoprotein receptor (LDLr−/− mice) that are kept on a high fat diet (HFD), which is a well-known animal model of atherosclerosis. These mice were kept either on a standard chow diet as controls or fed a high fat diet for 12 weeks to increase the formation of atherosclerotic plaques. The group of mice on a high fat diet were divided into three different groups, that were treated with (1) a HFD only, (2) a HFD and Omega-3 acid ethyl esters (OMACOR) or (3) a HFD and Compound-02. OMACOR are ethyl esters of the omega-3 fatty acids eicosapentaenoic acid (EPA) and docosahexaenoic acid (DHA) that naturally occur in fish oil. The mice were treated with 4 g/kg/d of OMACOR based on an avergae diet intake of 4 g in a 25 g mouse. Compound-02 was given at a dose of 120 mg/kg/d based on an avergae diet intake of 4 g in a 25 g mouse. All groups included 10 mice (N=10).

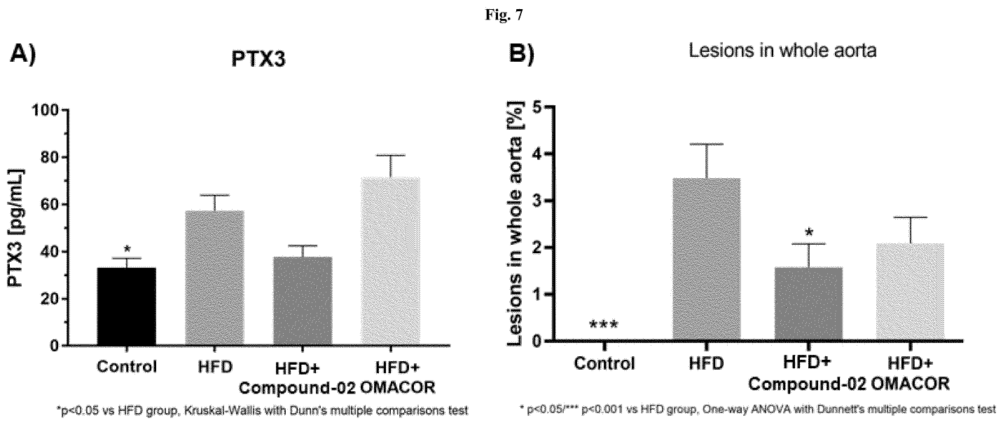

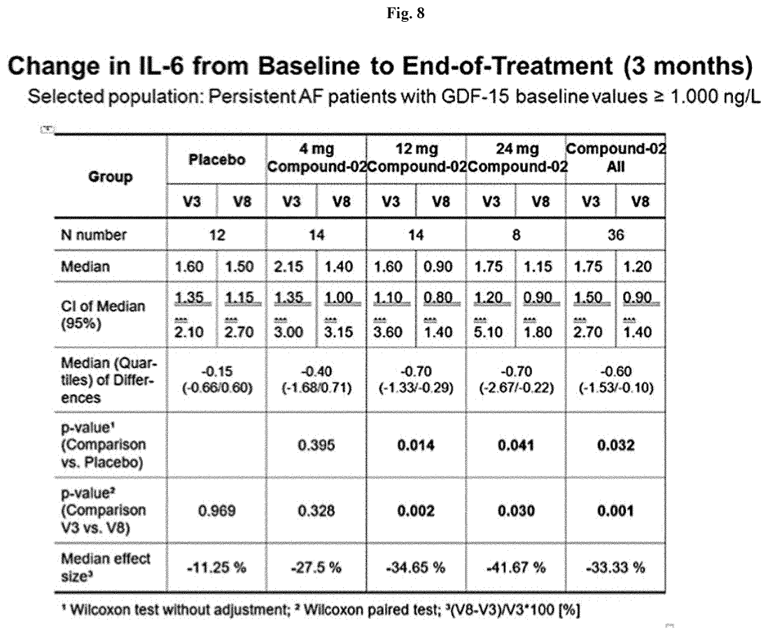

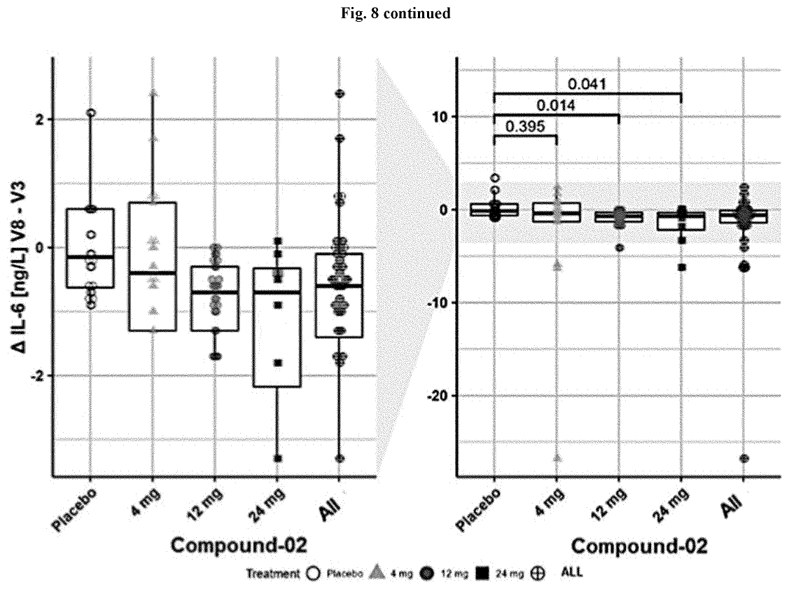

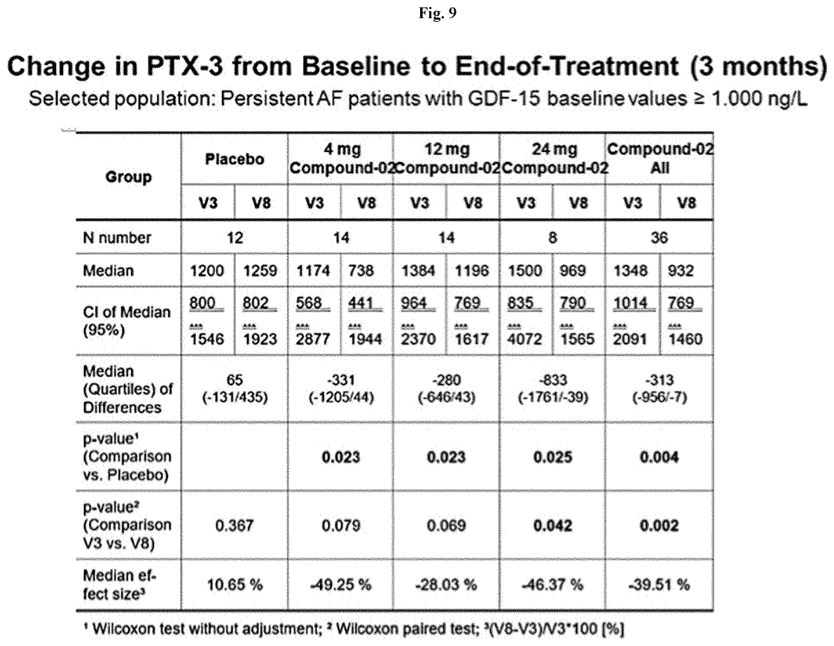

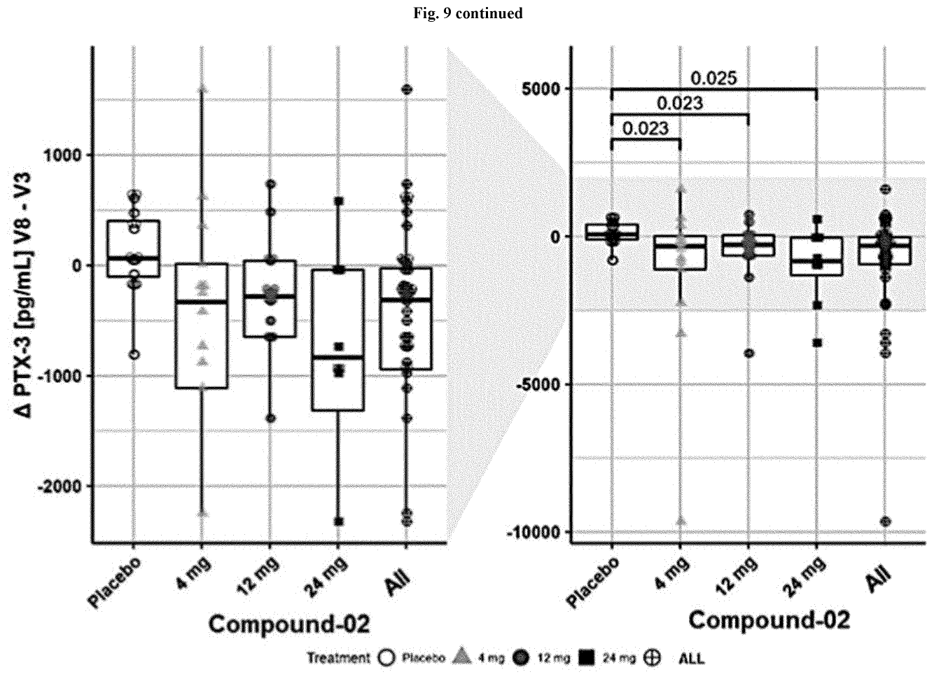

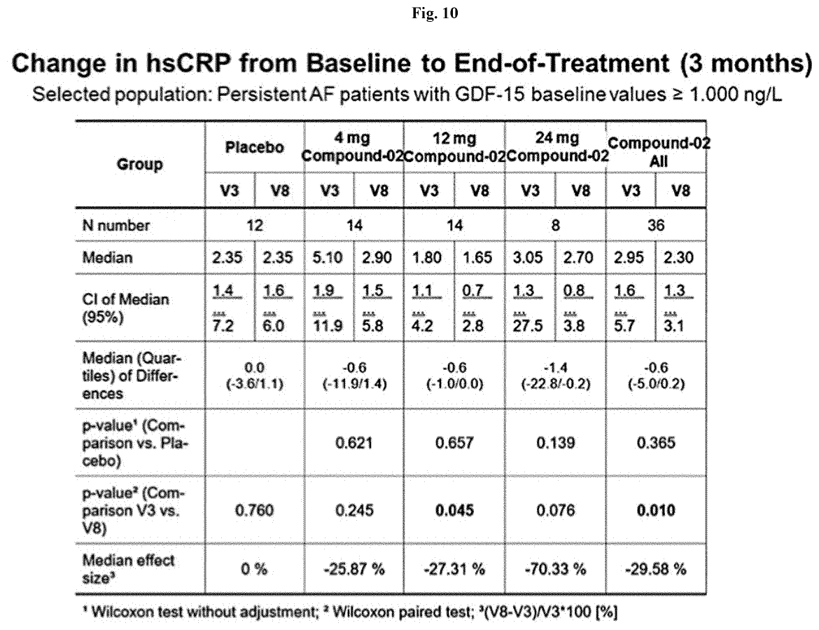

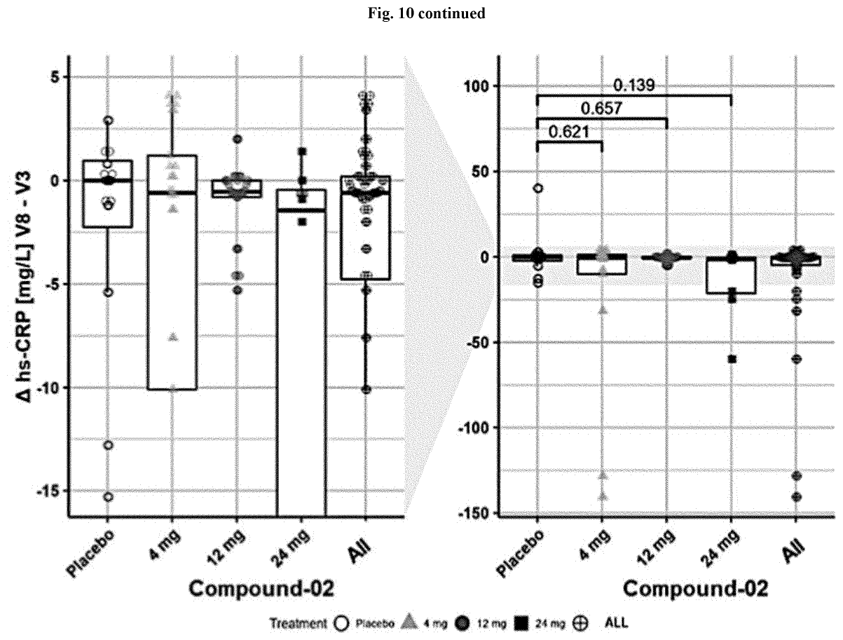

The level of PTX-3 were significantly increased in the mice fed a HFD compared to the control animals kept on a standard chow diet (see FIG. 7A) thereby confirming the increased risk caused by feeding a HFD. A similar increase was also observed in mice fed a HFD and OMACOR. In contrast the mice kept on a HFD supplemented with Compound-02 did not show an increase in PTX-3 levels and remained on the level of the control animals (see FIG. 7A).

The inventors observed also a significant reduction in lesions of the whole aorta in the mice treated with Compound-02consistent with the prevented increase of circulating PTX-3 levels observed in these mice (see FIG. 7B). The lesion size in whole aorta was reduced by about 55% in mice that received Compound-02 compared to those fed a HFD only.

The reduction of the biomarker of cardiac inflammation, PTX-3, in response to treatment with Compound-02 that was already observed in the clinical study of Example 2, was also observed in an animal model of atherosclerosis. Furthermore, treatment with Compound-02 did not only reduce the level of the biomarker but more importantly reduced the lesion size and therefore demonstrated therapeutic activity for atherosclerosis.

Atherosclerosis is the most common underlying mechainsm of coronary artery disease and peripheral artery disease.

### Example 4: Reduction of Cardiovascular Risk in Coronary Artery Disease Patients

In addition to the observed reduction in GDF-15, and PTX-3 levels Compound-02 further lowered the levels of interleukin-6 (IL-6) in the clinical study reported in Example 2. It was recently shown that reduction of IL-6 levels by pharmacological intervention in patients suffering from coronary artery disease (CAD) results in reduced cardiovascular event rates, independent of lipid lowering (Ridker P M et al., Eur Heart J 2018, 39:3499-3507). In the CANTOS study 4833 patients suffering from CAD were treated with the anti-IL-1β antibody Canakinumab that modulates the IL-6 pathway and thereby reduces circulating IL-6 levels.

Patients of CANTOS study with reduced circulating IL-6 levels demonstrated a 32% reduction in major adverse cardiac events (MACE) compared to the placebo group. The CANTOS study is therefore a clinical proof of concept study that lowering IL-6 levels in CAD patients provides a significant therapeutic benefit. Of note this effect of IL-6 lowering was found to be independent of any lipid lowering effects.

The significant reduction of IL-6 levels by 25-40% in response to treatment with Compound-02 in the clinical study of Examples therefore indicates that Compound-02 provides therapeutic benefit to CAD patients by at least reducing MACE rates.

### Example 5: Treatment with Compound-02 Lowers the Risk of Heart Failure

Further analysis of the patient subgroup with GDF-15 plasma concentration ≥1000 ng/L of the clinical study of Example 2 revealed the reduction of a marker for heart failure in response to lowering GDF-15 levels by treatment with Compound-02.

Specifically, the Compound-02 mediated lowering of GDF-15 plasma concentration is correlated with lowering of N-terminal pro-B-type natriuretic peptide (NT-proBNP) the current gold standard biomarker in heart failure (see McKie et al., J Am Coll Cardiol, 2016 Dec. 6; 68(22):2437-2439). A timepoint comparison between baseline (V3) and end of treatment (V8) showed a correlation of GDF-15 levels and NT-proBNP (see Table 3 below).

TABLE 3
Timepoint comparison baseline (V3) vs. end of treatment
(V8); GDF15 ≥ 1.000 ng/L: Compound-02, all dosed groups
together; Spearman Rank Correlations delta V8-V3 (n = 26)

| | hs-CRP | IL-6 | GDF15 | HbA1c | PTX-3 | HDL/LDL |
|---|---|---|---|---|---|---|
| | V8-V3 | V8-V3 | V8-V3 | V8-V3 | V8-V3 | V8-V3 |
| Var | [mg/L] | [ng/L] | [ng/L] | [%] | [pg/mL] | [mmol/L] |
| hs-CRP | 1 | | | | | |
| V8-V3 | | | | | | |
| [mg/L] | | | | | | |
| IL-6 | 0.595 | 1 | | | | |
| V8-V3 | | | | | | |
| [ng/L] | | | | | | |
| GDF15 | 0.28 | 0.143 | 1 | | | |
| V8-V3 | | | | | | |
| [ng/L] | | | | | | |
| HbA1c | 0.145 | −0.099 | 0.612 | 1 | | |
| V8-V3 [%] | | | | | | |
| NT-proBNP | 0.065 | 0.347 | 0.399 | 0.071 | | |
| V8-V3 | | | | | | |
| [ng/L] | | | | | | |
| PTX-3 | 0.104 | 0.236 | 0.295 | 0.133 | 1 | |
| V8-V3 | | | | | | |
| [pg/mL] | | | | | | |
| HDL/LDL | 0.065 | −0.083 | −0.283 | −0.583 | −0.174 | 1 |
| V8-V3 | | | | | | |
| [mmol/L] | | | | | | |

### Example 6: Treatment with Compound-02 Improves Diabetes Mellitus

Further analysis of the patient subgroup with GDF-15 plasma concentrations ≥1000 ng/L revealed a therapeutic benefit of the lowering of GDF-15 levels mediated by Compound-02 for the treatment of Diabetes mellitus.

A timepoint comparison of baseline (V3) and end of treatment (V8) parameters revealed a correlation between the lowering of GDF-15 levels and that of HbA1c (see Table 4 below). Glycated hemoglobin is a product of glycation, whereby sugar is attached to hemoglobin. The formation of increased amounts of glycated hemoglobin (i.e. HbA1c) indicates the presence of excessive blood glucose. Due to the average lifespan of red blood cells of about three months, the amount of HbA1c reflects the average glucose level in the last 8-12 weeks. This is the most widely used marker used in diabetes management and decreased levels indicate an improvement in glycemic control, which is required to reduce long term effects of Diabetes. The observed decrease in HbA1c therefore indicates a therapeutic benefit of lowering GDF-15 levels by treatment with Compound-02.

The therapeutic benefit in the treatment of Diabetes Mellitus with Compound-02 is further supported by the finding that important biomarkers of systemic chronic inflammation and vascular diseases (hs-CRP, IL-6 and PTX-3) are reduced by lowering of GDF-15 levels in response to treatment with Compound-02.

TABLE 4
Timepoint comparison baseline (V3) vs. end of treatment
(V8); GDF15 ≥ 1.000 ng/L: Compound-02, all dosed groups
together; Spearman Rank Correlations delta V8-V3 (n = 15)

| | hs-CRP | Triglyceride | IL-6 | GDF15 | HbA1c | PTX-3 | HDL/LDL |
|---|---|---|---|---|---|---|---|
| | V8-V3 | V8-V3 | V8-V3 | V8-V3 | V8-V3 | V8-V3 | V8-V3 |
| Var | [mg/L] | [mmol/L] | [ng/L] | [ng/L] | [%] | [pg/mL] | [mmol/L] |
| hs-CRP | 1 | | | | | | |
| V8-V3 [mg/L] | | | | | | | |
| Triglyceride | 0.364 | 1 | | | | | |
| V8-V3 | | | | | | | |
| [mmol/L] | | | | | | | |
| IL-6 | 0.556 | −0.145 | 1 | | | | |
| V8-V3 [ng/L] | | | | | | | |
| GDF15 | 0.354 | 0.314 | 0.3 | 1 | | | |
| V8-V3 [ng/L] | | | | | | | |
| HbA1c | 0.516 | 0.551 | 0.505 | 0.768 | 1 | | |
| V8-V3 [%] | | | | | | | |
| PTX-3 | 0.232 | −0.075 | 0.25 | 0.318 | 0.056 | 1 | |
| V8-V3 | | | | | | | |
| [pg/mL] | | | | | | | |
| HDL/LDL | −0.3 | −0.114 | −0.352 | −0.314 | −0.456 | −0.021 | 1 |
| V8-V3 | | | | | | | |
| [mmol/L] | | | | | | | |

### Example 7: Treatment with Compound-02 Improves Dyslipidemia

Further analysis of the patient subgroup with GDF-15 plasma concentrations ≥1000 ng/L revealed a therapeutic benefit of the lowering of GDF-15 levels mediated by Compound-02 for the treatment of Dsylipidemia.

Dyslipidemia is characterized by increased amount of lipids (e.g. triglycerides, cholesterol, in particular LDL) and is a risk factor in the development of cardiovascular diseases including coronary artery disease and peripheral artery disease as well as the metabolic syndrome.

An improvement of the HDL/LDL ratio in the patient subgroup with GDF-15 plasma concentrations ≥1000 ng/L was observed in response to lowering GDF-15 levels by treatment with Compound-02 (see Table 2 above). Furthermore, correlated triglyceride levels with the decreased GDF-15 levels in response to treatment with Compound-02 (see Table 4 above), which supports a therapeutic benefit in the treatment of Dyslipidemia.

1. A method of treating, reducing the risk of developing or preventing a disorder associated with an elevated GDF-15 plasma concentration comprising administering a compound of the general formula (I): 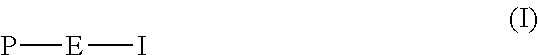
or a pharmaceutically acceptable salt thereof to a patient in need thereof, wherein
P is a group represented by the general formula (II): 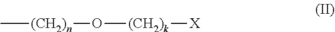
wherein
n is 0 or an integer from 3 to 8; and
k is 0, 1 or 2;
X represents CH₂OH, CH₂OAc, CH(O) or a group selected from the group consisting of: 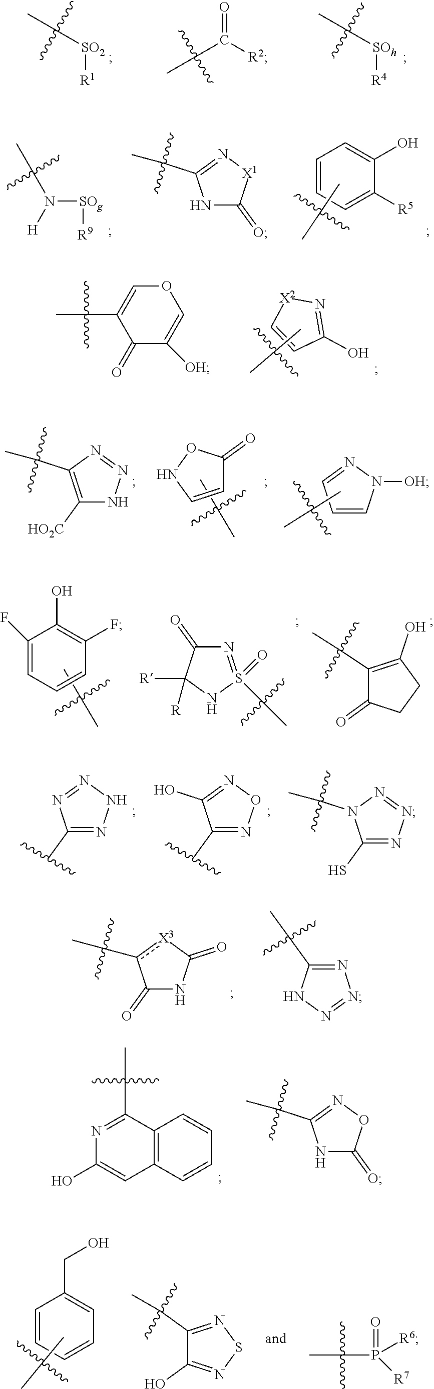
wherein
R and R′ each independently represents a hydrogen atom; or a C₁-C₆alkyl group which may be substituted with one or more fluorine or chlorine atom(s) or hydroxyl group(s);
R¹ represents a hydroxyl group, C₁-C₆alkoxy, NHCN, —NH(C₁-C₆alkyl), NH(C₃-C₆cycloalkyl), —NH(aryl), or O(C₁-C₆alkyldiyl)O(C=O)R¹¹; R¹¹ is a C₁-C₆alkyl group which is optionally substituted with one or more fluorine or chlorine atom(s); or a C₃-C₆cycloalkyl group which is optionally substituted with one or more fluorine or chlorine atom(s) or hydroxyl group(s);
R² represents —NHR³; —²⁰R²¹; —OR²²; —(OCH₂—CH₂)ᵢ—R²³; —C₃-C₁₀-heterocyclyl optionally substituted with one, two or three substituents independently selected from the group consisting of hydroxyl group, C₁-C₆alkoxy, C₁-C₆alkyl, and oxo; -(Xaa)o; a mono-, or disaccharide, or a derivative thereof, which is joined to —C(O) by an ester bond via the 1-O—, 3-O—, or 6-O-position of the saccharide;
or is selected from the group consisting of: 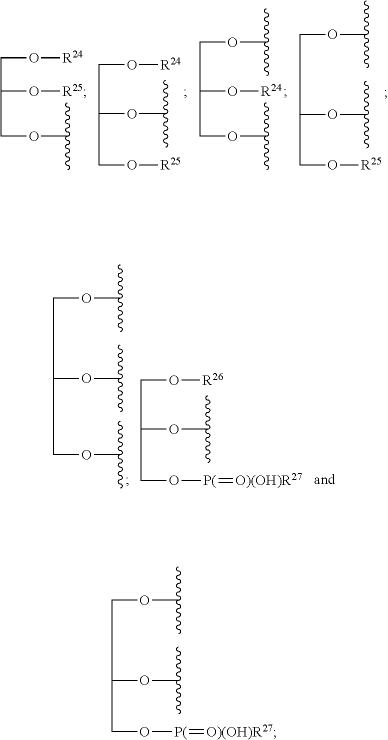
wherein
R³ represents (SO₂R³⁰); (OR³¹); —C₁-C₆alkanediyl(SO₂R³²); —C₁-C₆alkanediyl(CO₂H), an aryl group, a heteroaryl group, a cycloalkyl group or a heterocycloalkyl group, wherein the aryl group is optionally substituted with one, two or three substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), N(C₁-C₆)dialkyl, and —C(═O)OR⁵¹; wherein the heteroaryl group, is optionally substituted with one, two or three substituents independently selected from the group consisting of C₁-C₆alkyl, C₁-C₆alkoxy, C₁-C₆alkylthio, fluorine or chlorine atom, hydroxyl group, amino group, —NH(C₁-C₆alkyl), N(C₁-C₆)dialkyl and —C(═O)OR⁵¹; where the cycloalkyl group is optionally substituted with one, two or three substituents independently selected from the group consisting of C₁-C₆alkyl, C₁-C₆alkoxy, C₁-C₆alkylthio, fluorine or chlorine atom, hydroxyl group, amino group, —NH(C₁-C₆alkyl), —N(C₁-C₆)dialkyl, and —C(═O)OR⁵¹; and wherein the heterocycloalkyl group is optionally substituted with one, two or three substituents independently selected from the group consisting of C₁-C₆alkyl, C₁-C₆alkoxy, C₁-C₆alkylthio, fluorine or chlorine atom, hydroxyl group, amino group, —NH(C₁-C₆alkyl), N(C₁-C₆)dialkyl and —C(═O)OR⁵¹;
R³⁰ is a C₁-C₆alkyl, or an aryl group, wherein the C₁-C₆alkyl group is optionally substituted with —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, one, two or three fluorine or chlorine atoms, or a hydroxyl group; and wherein the aryl group is optionally substituted with one, two or three substituents independently selected from the group consisting of C₁-C₆alkyl, C₁-C₆alkoxy, C₁-C₆alkylthio, fluorine or chlorine atom, hydroxyl group, amino group, —NH(C₁-C₆alkyl), and —N(C₁-C₆)dialkyl;
R³¹ is a C₁-C₆alkyl group which is optionally substituted with one or more fluorine or chlorine atom(s) or hydroxyl group(s); or a C₃-C₆cycloalkyl group which is optionally substituted with one or more fluorine or chlorine atom(s) or hydroxyl group(s);
R³² is a C₁-C₆alkyl group which is optionally substituted with one or more fluorine or chlorine atom(s) or hydroxyl group(s); or a C₃-C₆cycloalkyl group which is optionally substituted with one or more fluorine or chlorine atom(s) or hydroxyl group(s);
R²⁰ and R²¹ each independently represents a hydrogen atom; a C₁-C₆alkyl group which may be substituted with one or more fluorine or chlorine atom(s) or hydroxyl group(s); a C₃-C₆cycloalkyl group which may be substituted with one or more fluorine or chlorine atom(s) or hydroxyl group(s); —C₁-C₆alkyldiyl(CO₂H) or together form a C₃-C₁₀-heterocycloalkyl which may be substituted with one or more C₁-C₆alkyl group(s), C₁-C₆alkoxy group(s), fluorine or chlorine atom(s) or hydroxyl group(s);
R²² is a hydrogen atom, a C₁-C₆alkyl group; or a C₃-C₆cycloalkyl group; wherein the C₁-C₆alkyl group or the C₃-C₆cycloalkyl group is optionally substituted with —NH₂, NH(C₁-C₆)alkyl, N(C₁-C₆)dialkyl, —NH(C₁-C₆)alkyldiyl- C₁-C₆alkoxy, one, two or three fluorine or chlorine atom(s), hydroxyl, or C₁-C₆alkoxy, an aralkyl group, a heteroalkyl group or a heteroalkylcycloalkyl group;
R²³ is —OH, —O(C₁-C₃)alkyl, or —N(C₁-C₃)dialkyl;
i is an integer of from 1 to 10;
R²⁴, R²⁵, and R²⁶ each independently represents a hydrogen atom; C(═O)C₁₁-C₂₁alkyl; or C(═O)C₁₁-C₂₁alkenyl;
R²⁷ represents OH; O(CH₂)₂NH₂, OCH₂—[CH(NH₂)(CO₂H)], O(CH₂)₂N(CH₃)₃; or 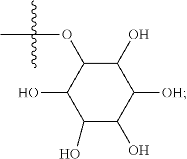 [SMILES: *OC1C(O)C(O)C(O)C(O)C1O]
Xaa represents Gly, a conventional D,L-, D- or L-amino acid, a non-conventional D,L-, D- or
L-amino acid, or a 2- to 10-mer peptide; and is joined to —C(═O) by an amide bond;
o is an integer of from 1 to 10;
R⁴ is selected from the group consisting of: 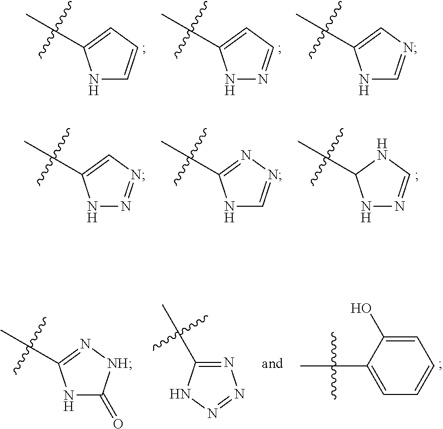
h is 0, 1, or 2;
R^{S} represents a hydrogen atom; a fluorine or chlorine atom; —CF₃; —C(═O)OR⁵¹;
—NHC(═O)R⁵²; —C(═O)NR⁵³R⁵⁴; or —S(O₂)OH;
R⁵¹ represents a hydrogen atom; a C₁-C₆alkyl group; or a C₃-C₆cycloalkyl group; wherein the C₁-C₆alkyl group or the C₃-C₆cycloalkyl group is optionally substituted with —NH₂, NH(C₁-C₆)alkyl, N(C₁-C₆)dialkyl, NH(C₁-C₆)alkyldiyl-C₁-C₆alkoxy, one, two or three fluorine or chlorine atom(s), hydroxyl, or C₁-C₆alkoxy;
R⁵², R⁵³ and R⁵⁴ each independently represents a C₁-C₆alkyl group which is optionally substituted with one or more fluorine or chlorine atom(s); a C₃-C₆cycloalkyl group which is optionally substituted with one or more fluorine or chlorine atom(s); or an aryl group which is optionally substituted with one, two or three substituents independently selected from the group consisting of C₁-C₆alkyl, C₁-C₆haloalkyl, C₁-C₆alkoxy, C₁-C₆alkylthio, fluorine or chlorine atom, hydroxyl group, amino group, —NH(C₁-C₆alkyl), N(C₁-C₆)dialkyl, and an oxo substituent;
R⁶ and R⁷ each independently represents a hydroxyl group; an —O(C₁-C₆)alkyl group, an —O(C₂-C₆)alkenyl group, a, —O(C₁-C₆)alkyldiylO(C═O)(C₁-C₆)alkyl group, or a —O(C₁-C₆)alkyldiylO(C=O)(C₂-C₆)alkenyl group; wherein the C₁-C₆alkyl group and the C₂-C₆alkenyl group may be substituted with NH₂, —NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆ alkylaminocarbonyl-, di(C₁-C₆)alkylaminocarbonyl-, or one, two or three fluorine or chlorine atom(s); or
R⁶ represents a hydroxyl group and R⁷ represents a group: 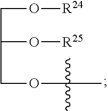
R⁹ represents C₁-C₆alkyl, or aryl; wherein the C₁-C₆alkyl is optionally substituted with —NH₂, NH(C₁-C₆)alkyl, N(C₁-C₆)dialkyl, NH(C₁-C₆)alkyldiyl-C₁-C₆alkoxy, one, two or three fluorine or chlorine atom(s), hydroxy, C₁-C₆alkoxy, aryl, aryloxy, C(═O)-aryl, C(═O)C₁-C₆alkoxy; and wherein the aryl group is optionally substituted with one, two or three substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl),
—N(C₁-C₆)dialkyl, and an oxo substituent;
g is 1 or 2;
X¹ represents an oxygen atom; sulfur atom; or NH;
X² represents an oxygen atom; sulfur atom; NH; or N(CH₃);
X³ represents an oxygen atom; sulfur atom; nitrogen atom; carbon atom; or C—OH; and the dashed line represents a carbon-carbon bond or a carbon-carbon double bond;
E is a group represented by the general formula (III) or (IV): 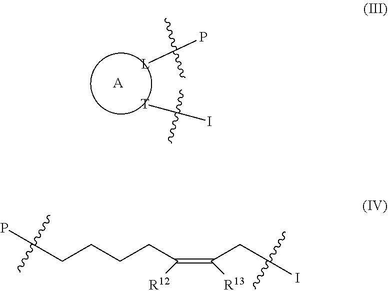
and wherein
ring A in formula (III) represents a 5-membered or 6-membered carbocyclic or heterocyclic ring containing at least one double bond, including an aromatic carbocyclic or heterocyclic ring, which can be substituted with one to three or one to four substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), and N(C₁-C₆)dialkyl; and L and T each independently represents a ring atom, wherein L and T are adjacent to another;
R¹² and R¹³ each independently represents a hydrogen atom, a fluorine atom, hydroxyl, —NH₂, C₁-C₆alkyl, C₁-C₆alkoxy, C(═O)-aryl, C(═O)C₁-C₆alkyl, or —SO₂(C₁-C₆alkyl); or —SO₂aryl; wherein any of the foregoing C₁-C₆alkyl, C₁-C₆alkoxy, or aryl are optionally substituted with one, two or three substituents independently selected from the group consisting of —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, fluorine or chlorine atom, and hydroxyl; or R¹² and R¹³ are taken together to form a 5-membered or 6-membered ring, which ring is optionally substituted with one, two or three substituents independently selected from the group consisting of —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, fluorine or chlorine atom, and hydroxyl;
I is —(CH₂)m-Y, wherein
m is an integer of from 3 to 6, provided that m is an integer of from 3 to 5 when E is a group according to general formula (III);
Y represents —U—V-W-(CH₂)ₚ—(CH₃)_{q}, wherein p is an integer from 0 to 6; q is 0 or 1; U is absent or selected from the group consisting of CH, CH₂ and NR⁴⁰, with the proviso that U is only CH if it forms an epoxy group together with V and W; V is selected from the group consisting of —C(O)—, —C(O)—C(O)—, —O—, and —S—; W is selected from the group consisting of CH, CH₂ and NR⁴⁰ with the proviso that W is only CH if it forms an epoxy group together with U and V;
or Y represents a group selected from the group consisting of: 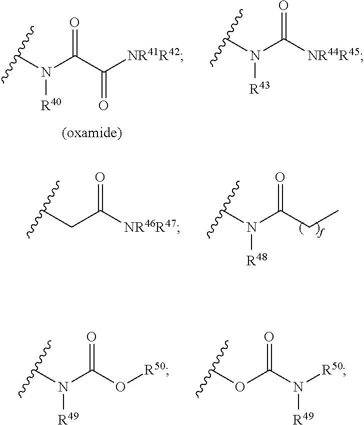
wherein
R⁴⁰, R⁴¹, R⁴³, R⁴⁴, R⁴⁶, R⁴⁸ and R⁴⁹ each independently represents a hydrogen atom, —C₁-C₆alkyl, —C₃-C₆cycloalkyl, —C₁-C₆alkoxy, C(═O)aryl, or C(═O)C₁-C₆alkyl, wherein any of the foregoing C₁-C₆alkyl, C₃-C₆cycloalkyl, C₁-C₆alkoxy, or aryl are optionally substituted with one, two or three substituents independently selected from the group consisting of —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, fluorine or chlorine atom, and hydroxy; or R⁴⁰ and R⁴¹, or R⁴³ and R⁴⁴, are taken together to form a 5-membered or 6-membered ring, which ring may be substituted with one, two or three substituents independently selected from the group consisting of —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, fluorine or chlorine atom, and hydroxyl;
R⁴², R⁴⁵, R⁴⁷ and R⁵⁰ each independently represents a —C₁-C₃alkyl, wherein the C₁-C₃alkyl may be substituted with one, two or three substituents independently selected from the group consisting of —NH₂, —NH(C₁-C₃)alkyl, N(C₁-C₃)dialkyl, C₁-C₃alkylcarbonyloxy-, C₁-C₃alkoxycarbonyloxy-, C₁-C₃alkylcarbonylthio-, C₁-C₃alkylaminocarbonyl-, di(C₁-C₃)alkylaminocarbonyl-, fluorine or chlorine atom, and hydroxyl; or R⁴⁰ and R⁴¹; R⁴³ and R⁴⁴; R⁴⁹ and R⁵⁰ are taken together to form a 5-membered or 6-membered ring, which ring may be substituted with one, two or three substituents independently selected from the group consisting of —NH₂, NH(C₁-C₆)alkyl, N(C₁-C₆)dialkyl, C₁-C₆alkylcarbonyloxy-, C₁-C₆alkoxycarbonyloxy-, C₁-C₆alkylcarbonylthio-, C₁-C₆alkylaminocarbonyl-, di(C₁-C₆)alkylaminocarbonyl-, fluorine or chlorine atom, and hydroxyl;
f is an integer of from 0 to 2;
with the proviso that
when X does not comprise a —C(═O)O-motif with the carbonyl carbon in alpha or beta position to the oxygen atom of general formula (II), Y is an oxamide, a carbamate or a carbamide.

2. The method according to claim 1,
with the proviso that
when n is 3, 5, 6, 7 or 8, k is 1 and E is a group according to general formula (III) or general formula (IV), wherein each of R¹² and R¹³ is a hydrogen atom;
P represents a group: 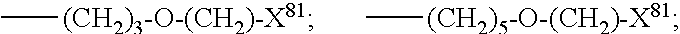
wherein
X⁸¹ represents a group selected from the group consisting of: 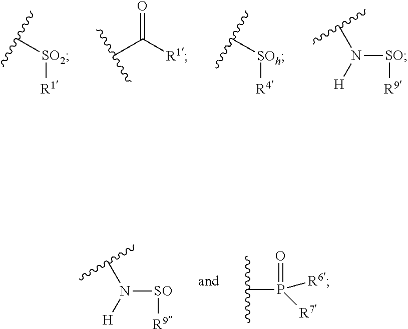
R^{1′} is defined as R¹ above;
R^{2′} represents —NHR^{3′}; —OR^{22′}; —(OCH₂—CH₂)ᵢ—R²³; a mono-, or disaccharide, or a derivative thereof, which is joined to —C(═O) by an ester bond via the 1-O—, 3-O—, or 6-O-position of the saccharide;
or wherein R² is selected from the group consisting of: 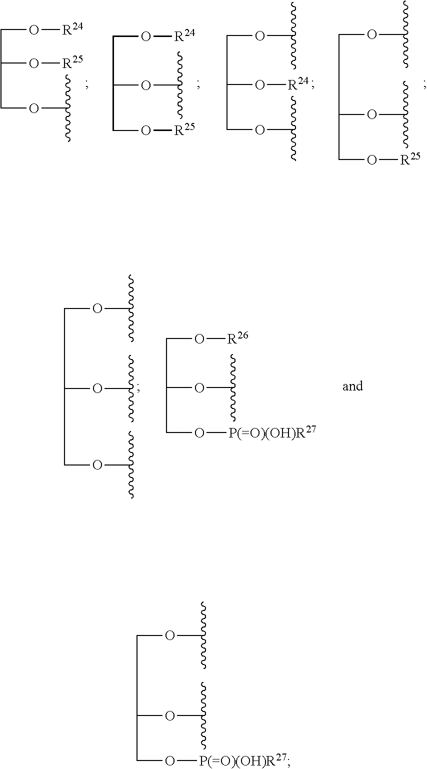
wherein
R^{3′} represents (SO₂R³⁰); (OR³¹); —C₁-C₆alkanediyl(SO₂R³²); or —C₂-C₆alkanediyl(CO₂H);
R^{22′} is a hydrogen or a C₃-C₆cycloalkyl group, which is optionally substituted with —NH₂, NH(C₁-C₆)alkyl, N(C₁-C₆)dialkyl, —NH(C₁-C₆)alkyldiyl- C₁-C₆alkoxy, one, two or three fluorine or chlorine atom(s), hydroxy, or C₁-C₆alkoxy;
R²³ and i are as defined above;
R²⁴, R²⁵, R²⁶, and R²⁷ are as defined above;
R^{4′} is defined as R⁴ above; and h is defined as above;
R^{6′} and R^{7′} are defined as R⁶ and R⁷ above;
R^{9′} is defined as R⁹ above; R^{9′} represents aryl which is optionally substituted with one, two or three substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), N(C₁-C₆)dialkyl, and an oxo substituent.

3. The method according to claim 1, wherein X is 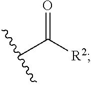
wherein R² is —OR²²; —(OCH₂—CH₂)ᵢ—R²³; a mono-, or disaccharide, or a derivative thereof, which is joined to —C(═O) by an ester bond via the 1-O—, 3-O—, or 6-O-position of the saccharide;
or wherein R² is selected from the group consisting of: 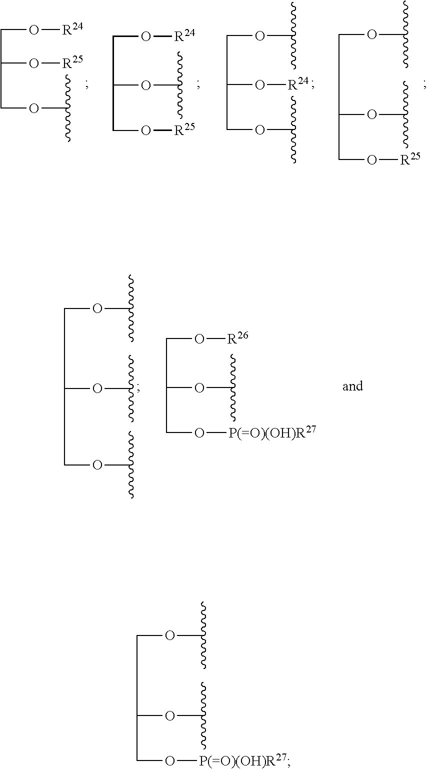
wherein R²³ and i are as defined above;
and wherein R²², and R²³ to R²⁷ are as defined in claim 1.

4. The method according to claim 1, wherein X is —C(═O)OH or a suitable salt of the carboxylic acid.

5. The method according to claim 1, wherein Y is one of the oxamides defined according to claim 1.

6. The method according to claim 1, wherein X is 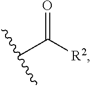
wherein R² is —OR²²; —(OCH₂—CH₂)ᵢ—R²³; a mono-, or disaccharide, or a derivative thereof, which is joined to —C(═O) by an ester bond via the 1-O—, 3-O—, or 6-O-position of the saccharide;
or wherein R² is selected from the group consisting of: 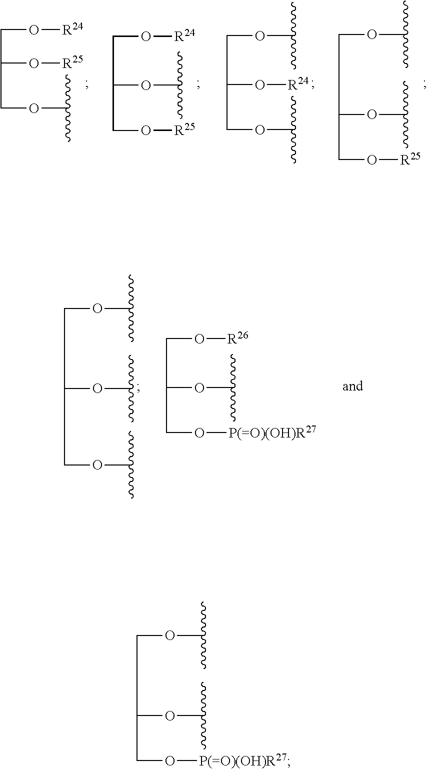
wherein R²², R²³ to R²⁷ and i are as defined in claim 1, and wherein Y is one of the oxamides defined according to claim 1.

7. The method according to claim 1, wherein X is C(═O)OH, and Y is one of the oxamides defined according to claim 1.

8. The method according to claim 1, with the formula (V) 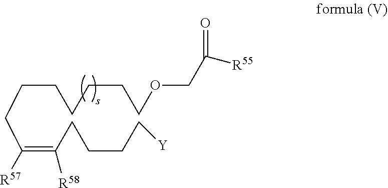
wherein
R⁵⁵ represents —OH; —OR²²; —(OCH₂—CH₂)ᵢ—R²³; a mono-, or disaccharide, or a derivative thereof, which is joined to —C(═O) by an ester bond via the 1-O—, 3-O—, or 6-O-position of the saccharide;
R²², R²³ and i are as defined in claim 1;
Y represents a group selected from the group consisting of: 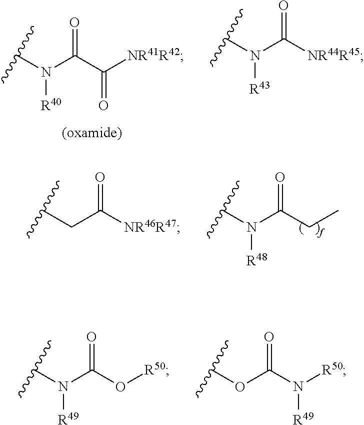
wherein R⁴⁰ to R⁵⁰ are defined in claim 1;
R⁵⁷ and R⁵⁸ are hydrogen; or form together a five- or six-membered ring, optionally substituted with one to three or one to four substituents independently selected from the group consisting of C₁-C₆alkyl, C₁-C₆alkoxy, C₁-C₆alkylthio, fluorine or chlorine atom, hydroxyl group, amino group, NH(C₁-C₆alkyl), N(C₁-C₆)dialkyl, and an oxo substituent;
s is 0, 1 or 2, with the proviso that s is 0 if R⁵⁷ and R⁵⁸ form together a five- or six-membered ring;
the double bond in formula (V) represents a double carbon-carbon bond in cis- configuration, if R⁵⁷ and R⁵⁸ are hydrogen, or this double bond is part of a five- or six-membered ring formed together by R⁵⁷ and R⁵⁸.

9. The method according to claim 8, wherein
R⁵⁵ represents —OH or —(OCH₂—CH₂)ᵢ—R²³; i is 2 to 4;
Y is an oxamide, a carbamide or a carbamate,
R⁵⁷ and R⁵⁸ are both H, or together form a substituted or non-substituted five- or six-membered aromatic ring; and
s is 1 or s is 0 if R⁵⁷ and R⁵⁸ together form a substituted or non-substituted five- or six-membered aromatic ring.

10. The method according to claim 1, wherein the compound is selected from the group consisting of: 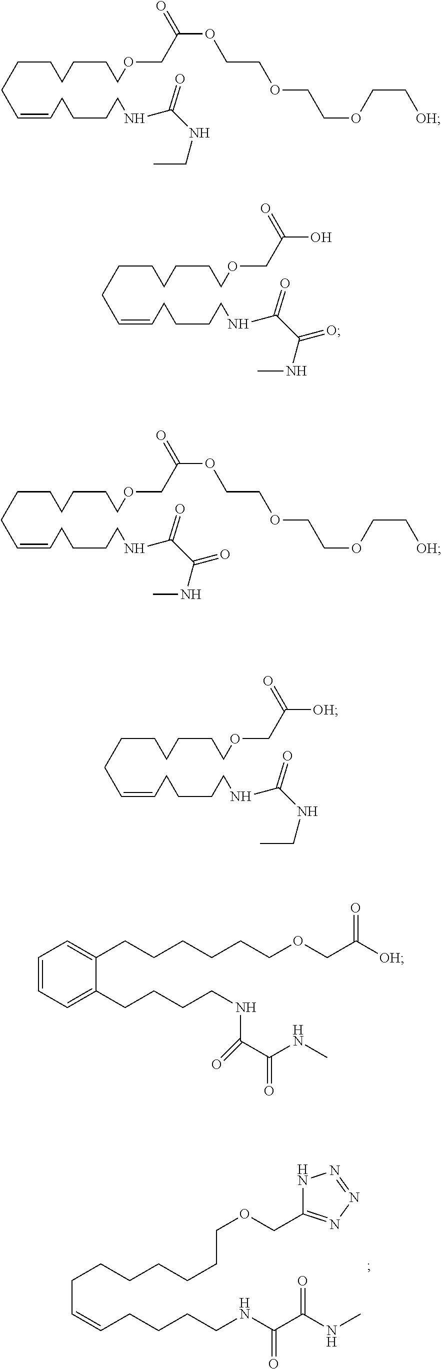 [SMILES: CCNC(=O)NCCCC/C=C\CCCCCCCOCC(=O)OCCOCCOCCO ; CNC(=O)C(=O)NCCCC/C=C\CCCCCCCOCC(=O)O ; CNC(=O)C(=O)NCCCC/C=C\CCCCCCCOCC(=O)OCCOCCOCCO ; CCNC(=O)NCCCC/C=C\CCCCCCCOCC(=O)O ; CNC(=O)C(=O)NCCCCc1ccccc1CCCCCCOCC(=O)O ; CNC(=O)C(=O)NCCCC/C=C\CCCCCCCOCc1nnn[nH]1] 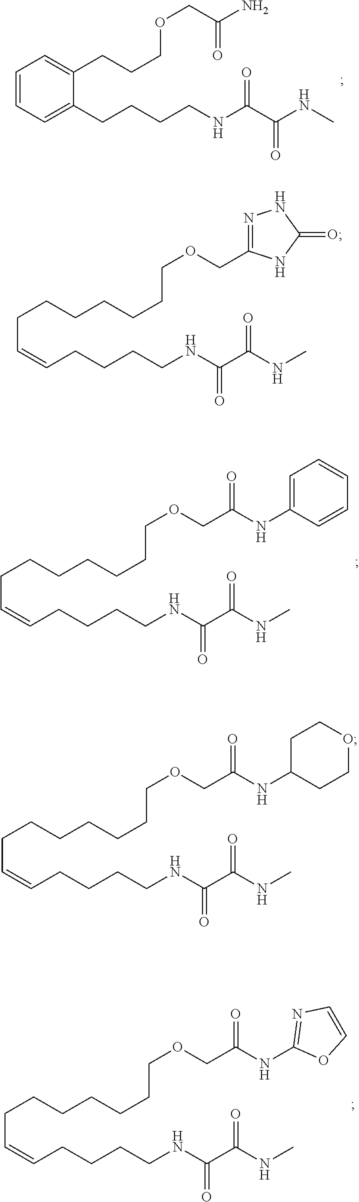 [SMILES: CNC(=O)C(=O)NCCCCc1ccccc1CCCOCC(N)=O ; CNC(=O)C(=O)NCCCC/C=C\CCCCCCCOCc1n[nH]c(=O)[nH]1 ; CNC(=O)C(=O)NCCCC/C=C\CCCCCCCOCC(=O)Nc1ccccc1 ; CNC(=O)C(=O)NCCCC/C=C\CCCCCCCOCC(=O)NC1CCOCC1 ; CNC(=O)C(=O)NCCCC/C=C\CCCCCCCOCC(=O)Nc1ncco1] 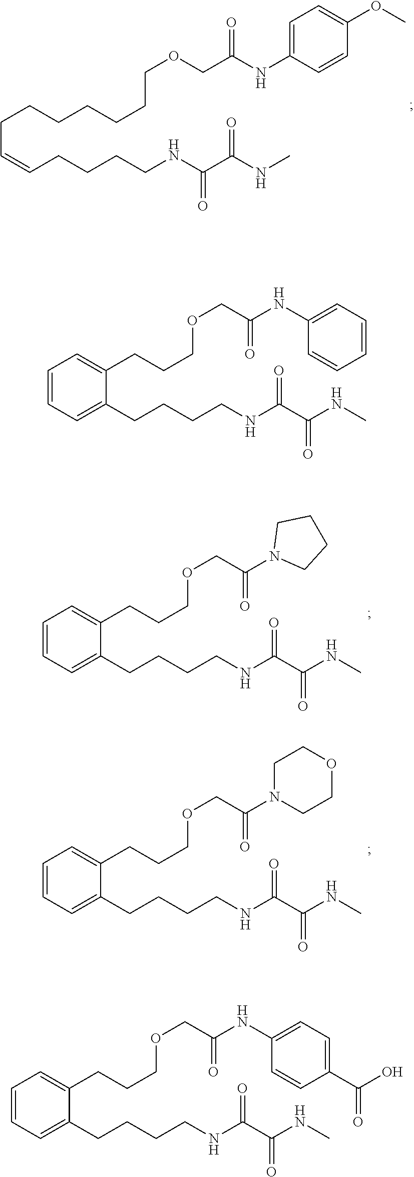 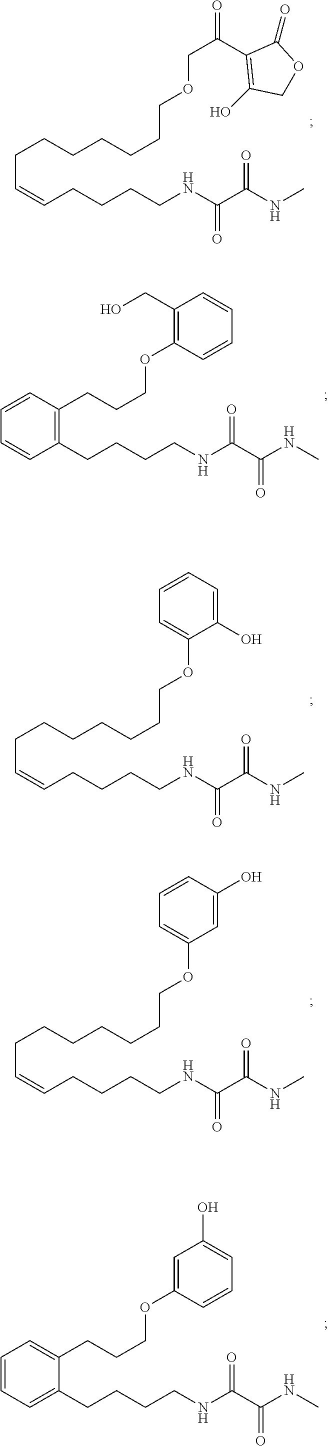 [SMILES: CNC(=O)C(=O)NCCCC/C=C\CCCCCCCOCC(=O)C1=C(O)COC1=O ; CNC(=O)C(=O)NCCCCc1ccccc1CCCOc1ccccc1CO ; CNC(=O)C(=O)NCCCC/C=C\CCCCCCCOc1ccccc1O ; CNC(=O)C(=O)NCCCC/C=C\CCCCCCCOc1cccc(O)c1 ; CNC(=O)C(=O)NCCCCc1ccccc1CCCOc1cccc(O)c1] 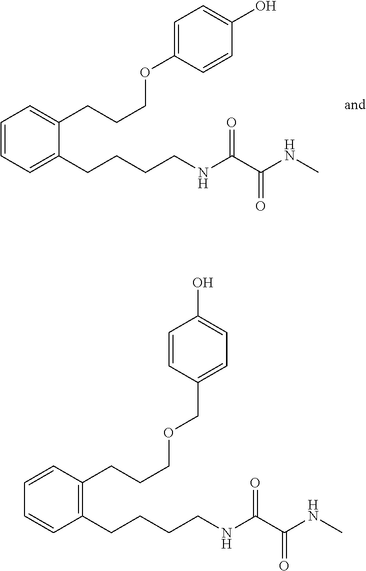 [SMILES: CNC(=O)C(=O)NCCCCc1ccccc1CCCOc1ccc(O)cc1 ; CNC(=O)C(=O)NCCCCc1ccccc1CCCOCc1ccc(O)cc1]
or a pharmaceutically acceptable salt thereof.

11. The method according to claim 1, with the formula (VI) 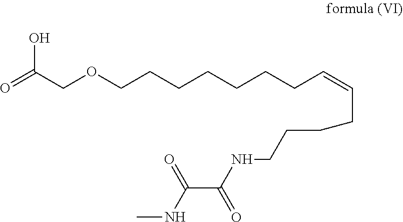 [SMILES: CNC(=O)C(=O)NCCCC/C=C\CCCCCCCOCC(=O)O]
or a pharmaceutically acceptable salt thereof.

12. The method according to claim 1, wherein the disorder-associated with an elevated GDF-15 plasma concentration is a cardiovascular disease.

13. The method according to claim 1, wherein the disorder associated with an elevated GDF-15 plasma concentration is a metabolic disease.

14. The method according to claim 12, wherein the cardiovascular disease is selected from atrial fibrillation, bleeding risk associated with atrial fibrillation, a coronary artery disease (CAD), angina, myocardial infarction, stroke, heart failure, hypertensive heart disease, rheumatic heart disease, cardiomyopathy, congenital heart disease, valvular heart disease, carditis, aortic aneurysm, peripheral artery disease, thromboembolic disease and venous thrombosis.

15. The method according to claim 13, wherein the metabolic disease is selected from diabetes mellitus, dyslipidemia, and metabolic syndrome.

16. The method according to claim 1, wherein k is 1.

17. The method according to claim 1, wherein X is

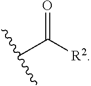

18. The method according to claim 1, wherein Y is an oxamide as defined in claim 1.

19. The method according to claim 1, wherein the GDF-15 plasma concentration is at least 1000 ng/L.

20. The method of claim 4, wherein the carboxylic acid is a free carboxylic acid.
